(19) United States
(12) Patent Application Publication
(10) Pub. No.: US 2024/0209361 A1
(43) Pub. Date: Jun. 27, 2024

(54) TREATING CANCER

(71) Applicants: The Johns Hopkins University, Baltimore, MD (US); Regents of the University of Minnesota, Minneapolis, MN (US)

(72) Inventors: Efrosini Kokkoli, Baltimore, MD (US); Clark C. Chen, Minneapolis, MN (US); Zachary Schneiderman, Baltimore, MD (US)

(21) Appl. No.: 18/288,457

(22) PCT Filed: Apr. 27, 2022
(86) PCT No.: PCT/US2022/026464

§ 371 Date: Oct. 26, 2023

Related U.S. Application Data
(60) Provisional application No. 63/180,995, filed on Apr. 28, 2021.

(51) Int. Cl. C12N 15/113; A61K 9/127; A61K 9/51; A61K 47/59; A61K 47/64; A61K 47/69; A61N 5/10; A61P 35/00

(52) U.S. Cl. CPC C12N 15/113; A61K 9/1272; A61K 9/5169; A61K 47/59; A61K 47/64; A61K 47/6911; A61N 5/10; A61P 35/00; C12N 15/1135; A61N 2005/1098; C12N 2310/113; C12N 2310/141; C12N 2320/31

(57) ABSTRACT

This document relates to methods and materials for treating cancer. For example, this document provides nanoparticles (e.g., liposomes and LNPs) encapsulating one or more microRNAs and/or one or more anti-microRNAs. In some cases, nanoparticles (e.g., liposomes and LNPs) encapsulating one or more microRNAs and/or one or more anti-microRNAs can be administered to a mammal (e.g., a human) having cancer to treat the mammal.

### CROSS-REFERENCE TO RELATED APPLICATIONS

This application claims the benefit of U.S. Patent Application Ser. No. 63/180,995, filed on Apr. 28, 2021. The disclosure of the prior application is considered part of (and is incorporated by reference in) the disclosure of this application.

### TECHNICAL FIELD

This document relates to methods and materials for treating cancer. For example, this document provides nanoparticles (e.g., liposomes and lipid nanoparticles (LNPs)) encapsulating one or more microRNAs and/or one or more anti-microRNAs). In some cases, nanoparticles (e.g., liposomes and LNPs) encapsulating one or more microRNAs and/or one or more anti-microRNAs can be administered to a mammal (e.g., a human) having cancer to treat the mammal.

### BACKGROUND INFORMATION

Glioblastoma (GBM) is recognized as the most common and invasive primary brain tumor (Adamson et al., Expert. Opin. Investig. Drugs, 18:1061-1083 (2009)). The gold standard first line therapy for GBM encompasses ionizing radiation (IR) and a DNA alkylating agent, temozolomide (TMZ). However, tumor progression and recurrence typically occur due to development of resistance to IR and TMZ, and thus GBM patients have a poor prognosis with a 5-year survival rate of 5.1% (Taylor et al., Front. Oncol., 9:963 (2019)).

Breast cancer is the most common cancer and the second most deadly cancer in women worldwide (Wahba & El-Hadaad, Cancer Biol. Med., 12:106-116 (2015)). Approximately 15-20% of all breast cancers are triple negative in the United States, lacking the commonly targeted estrogen and progesterone receptors, and do not demonstrate human epidermal growth factor receptor 2 (HER2) overexpression or amplification (Prakash et al., Front. Public Health 8:576964 (2020)). Common breast cancer hormonal therapies that target estrogen and progesterone receptors are not viable for triple negative breast cancer (TNBC) (Collignon et al., Breast Cancer (Dove Medical Press) 8:93-107 (2016)), nor are the numerous antibody, antibody drug conjugate, and kinase inhibiting therapeutics available for HER2 overexpressing cancers (Bredin et al., Semin. Oncol. 47:259-269 (2020)).

### SUMMARY

This document provides methods and materials for treating cancer (e.g., a central nervous system (CNS) cancer such as GBM or a breast cancer such as a TNBC). For example, this document provides nanoparticles (e.g., liposomes and LNPs) encapsulating one or more microRNAs (e.g., miR-603) and/or one or more anti-microRNAs (e.g., anti-miR-21) and including a targeting moiety. In some cases, nanoparticles (e.g., liposomes and LNPs) encapsulating one or more microRNAs (e.g., miR-603) and/or one or more anti-microRNAs (e.g., anti-miR-21) and including a targeting moiety can be administered to a mammal (e.g., a human) having cancer (e.g., a CNS cancer such as GBM or a breast cancer such as a TNBC) to treat the mammal.

As demonstrated herein, liposomes encapsulating miR-603 with polyethylenimine (miR-603/PEI complexes) can be functionalized with the peptide KSSPHSRNSGSGSGSGSGRGDSP (SEQ ID NO:5) to target and deliver the miR-603/PEI complexes to GBM cells such as GBM stem-like cells. Also as demonstrated herein, administering miR-603/PEI complexes to GBM cells such as GBM stem-like cells can sensitize the cells to radiation therapy.

Also as demonstrated herein, liposomes encapsulating anti-miR-21 with polyethylenimine (anti-miR-21/PEI complexes) can be functionalized with the peptide KSSPHSRNSGSGSGSGSGRGDSP (SEQ ID NO:5) to target and deliver the anti-miR-21/PEI complexes to GBM cells. For example, LNPs encapsulating miR-603 or anti-miR-21 can be functionalized with the peptide KSSPHSRNSGSGSGSGSGRGDSP (SEQ ID NO:5) to target and deliver the miR-603 or the anti-miR-21 to cancer cells (e.g., GBM cells and TNBC cells) to decrease the proliferation of the cancer cells.

Having the ability to specifically target GBM cells and/or TNBC cells within a mammal (e.g., a human) as described herein (e.g., by administering nanoparticles (e.g., liposomes and LNPs) encapsulating one or more microRNAs (e.g., miR-603) and/or one or more anti-microRNAs (e.g., anti-miR-21) and including a targeting moiety) provides a unique and unrealized opportunity to target cancer cells that are often treatment-resistant, and can sensitize the cancer cells to radiation therapy and/or decrease proliferation of the cancer cells.

In general, one aspect of this document features nanoparticles encapsulating miR-603, where the nanoparticles include a targeting moiety comprising a polypeptide including the amino acid sequence set forth in SEQ ID NO:5. The nanoparticle can be a liposome, an LNP, an extracellular vesicle, a polymersome, a polymeric nanoparticle, or a micelle. The peptide can be consists essentially of the amino acid sequence set forth in SEQ ID NO:5. The peptide can consists of the amino acid sequence set forth in SEQ ID NO:5. The targeting moiety can be conjugated to a hydrophobic tail having the structure (C₁₆)₂-Glu-C₂.

In another aspect, this document features nanoparticles encapsulating anti-miR-21, where the nanoparticles include a targeting moiety comprising a polypeptide including the amino acid sequence set forth in SEQ ID NO:5. The nanoparticle can be a liposome, an LNP, an extracellular vesicle, a polymersome, a polymeric nanoparticle, or a micelle. The peptide can be consists essentially of the amino acid sequence set forth in SEQ ID NO:5. The peptide can consists of the amino acid sequence set forth in SEQ ID NO:5. The targeting moiety can be conjugated to a hydrophobic tail having the structure (C₁₆)₂-Glu-C₂.

In another aspect, this document features methods for treating a mammal having cancer. The methods can include, or consist essentially of, administering a composition comprising nanoparticles encapsulating miR-603 and/or nanoparticles encapsulating anti-miR-21, where the nanoparticle includes a targeting moiety comprising a polypeptide including the amino acid sequence set forth in SEQ ID NO:5 to the mammal. The mammal can be a human. The cancer can be a glioblastoma, a diffuse intrinsic pontine glioma, an astrocytoma, an oligodendroglioma, an oligoastrocytoma, an ependymoma, a medulloblastoma, a meningioma, a breast cancer, a colon cancer, a liver cancer, a pancreatic cancer, or a prostate cancer. The method also can include subjecting the mammal to radiation therapy. The method also can include administering doxorubicin, cyclophosphamide temozolomide, cisplatin, 5FU, paclitaxel, gemcitabine, or any combinations thereof to the mammal.

In another aspect, this document features methods for sensitizing cancer cells to radiation therapy. The methods can include, or consist essentially of, administering a composition comprising nanoparticles encapsulating miR-603 and/or nanoparticles encapsulating anti-miR-21, where the nanoparticle includes a targeting moiety comprising a polypeptide including the amino acid sequence set forth in SEQ ID NO:5 to a mammal having cancer. The mammal can be a human. The cancer can be a glioblastoma, a diffuse intrinsic pontine glioma, an astrocytoma, an oligodendroglioma, an oligoastrocytoma, an ependymoma, a medulloblastoma, a meningioma, a breast cancer, a colon cancer, a liver cancer, a pancreatic cancer, or a prostate cancer. The method also can include subjecting the mammal to the radiation therapy. The method also can include administering doxorubicin, cyclophosphamide temozolomide, cisplatin, 5FU, paclitaxel, gemcitabine, or any combinations thereof to the mammal.

In another aspect, this document features methods for treating a mammal having cancer. The methods can include, or consist essentially of, administering a composition comprising nanoparticles encapsulating miR-603 and/or nanoparticles encapsulating anti-miR-21, where the nanoparticle includes a targeting moiety comprising a polypeptide including the amino acid sequence set forth in SEQ ID NO:5 to a mammal having cancer; and subjecting the mammal to radiation therapy. The mammal can be a human. The cancer can be a glioblastoma, a diffuse intrinsic pontine glioma, an astrocytoma, an oligodendroglioma, an oligoastrocytoma, an ependymoma, a medulloblastoma, a meningioma, a breast cancer, a colon cancer, a liver cancer, a pancreatic cancer, or a prostate cancer. The method also can include administering doxorubicin, cyclophosphamide temozolomide, cisplatin, 5FU, paclitaxel, gemcitabine, or any combinations thereof to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Representative bright field microscopy image of a GBM-CCC-001 neurosphere.

FIGS. 2A-2C. Cryo-TEM images of miR-603/PEI complexes at N:P=6 (FIG. 2A), empty PR_b liposomes (FIG. 2B), and PR_b liposomes encapsulating miR-603/PEI complexes at N:P=6 (FIG. 2C).

FIG. 3. α₅β₁ integrin expression on patient-derived glioblastoma stem-like cells. Data are presented as mean±SD (n=3).

FIGS. 4A-4D. FIG. 4A) Representative α₅β₁ integrin expression on GBM-CCC-001 cells. FIG. 4B) Cell association, via flow cytometry, of non-targeted liposomes and PR_b-functionalized liposomes with GBM-CCC-001 cells after 48 hours at 37° C. Data are presented as mean±SD (n=2). Student's t-test analysis was used to determine significance, * p<0.05. Confocal microscopy images of non-targeted liposomes (FIG. 4C) and PR_b-functionalized liposomes (FIG. 4D) incubated for 48 hours at 37° C. with GBM-CCC-001 cells. Liposomes are shown in green, cell membranes are shown in red, and nuclei are shown in blue.

FIG. 5. Cell association, via flow cytometry, of non-targeted liposomes and PR_b-functionalized liposomes with patient-derived glioblastoma stem-like cells after 48 hours at 37° C. Data are presented as mean±SD (n=2). Student's t-test analysis was used to determine significance, * p<0.05.

FIG. 6. Cell association, via flow cytometry, of PR_b-functionalized liposomes with GBM-CCC-001 cells or cells with their surface integrins blocked by free PR_b peptide for 1 hour at 37° C. before incubating the cells with PR_b liposomes for an additional 1 hour at 37° C. Data are presented as mean±SD (n=3). Student's t-test analysis was used to determine significance, ** p <0.01.

FIGS. 7A-7D. Confocal microscopy images of liposomes incubated with cells for 48 hours at 37° C. Top row shows GBM-CCC-002 cells with non-targeted liposomes (FIG. 7A) and PR_b-functionalized liposomes (FIG. 7B). Bottom row shows GBM-CCC-003 cells with non-targeted liposomes (FIG. 7C) and PR_b-functionalized liposomes (FIG. 7D). Liposomes are shown in green, cell membranes are shown in red, and nuclei are shown in blue.

FIGS. 8A-8B. mRNA levels of (FIG. 8A) miR-603 and (FIG. 8B) IGF1 and IGF1R relative to PBS control determined by qRT-PCR in GBM-CCC-001 cells following exposure to indicated treatments for 48 hours at 37° C. Data are presented as mean±SD (n=3). P-values from one-way ANOVA with Tukey's HSD post-hoc analysis can be found in Tables 3A, 3B, and 4.

FIGS. 9A-9C. FIG. 9A) Schematic representation of the limiting dilution assay. Limiting dilution analysis demonstrating the clonogenic potential of GBM-CCC-001 cells after exposure to different liposomal formulations for either 24 hours (FIG. 9B) or 48 hours (FIG. 9C) before IR treatment. Groups without IR treatment were also used. For every group, 24 and 16 wells per cell dose were used for the 24- and 48-hour treatment, respectively. Statistical analysis was performed using the ELDA software and p-values can be found in Tables 5 and 6.

FIG. 10. U87 GBM cells were incubated with PR_b functionalized liposomes encapsulating anti-miR-21/PEI complexes (180 nM) for 48 hours. Expression or miR-21 was evaluated with RT-qPCR via the Qiagen kit specifically aimed at miR detection. Statistical significance was assessed between treated and untreated cells (control) using pairwise t-test; * P<0.01.

FIGS. 11A and 11B. Cryo-TEM images of PR_b (mix) LNPs encapsulating (FIG. 11A) anti-miR-21, or (FIG. 11B) mir-603.

FIG. 12. PANC 10.05 human pancreatic cancer cells were incubated with PR_b (mix) LNPs encapsulating anti-miR-21 (180 nM) for 48 or 72 hours. Expression or miR-21 was evaluated with RT-qPCR via the Qiagen kit specifically aimed at miR detection.

### DETAILED DESCRIPTION

This document provides methods and materials for treating cancer (e.g., a CNS cancer such as GBM or a breast cancer such as a TNBC). For example, this document provides nanoparticles (e.g., liposomes and LNPs) encapsulating one or more microRNAs (e.g., miR-603) and/or one or more anti-microRNAs (e.g., anti-miR-21) and including a targeting moiety. In some cases, nanoparticles (e.g., liposomes and LNPs) encapsulating one or more microRNAs (e.g., miR-603) and/or one or more anti-microRNAs (e.g., anti-miR-21), and including a targeting moiety can be administered to a mammal (e.g., a human) having cancer (e.g., a CNS cancer such as GBM or a breast cancer such as a TNBC) to treat the mammal. For example, nanoparticles (e.g., liposomes and LNPs) encapsulating miR-603 and including a targeting moiety can be administered to a mammal (e.g., a human) having cancer (e.g., a CNS cancer such as GBM or a breast cancer such as a TNBC) to increase expression of miR-603 in cancer cells within the mammal. For example, nanoparticles (e.g., liposomes and LNPs) encapsulating anti-miR-21 and including a targeting moiety can be administered to a mammal (e.g., a human) having cancer (e.g., a CNS cancer such as GBM or a breast cancer such as a TNBC) to reduce or eliminate expression of miR-603 in cancer cells within the mammal.

A nanoparticle provided herein (e.g., a nanoparticle encapsulating one or more microRNAs such as miR-603 and/or one or more anti-microRNAs such as anti-miR-21 and including a targeting moiety) can be any appropriate type of nanoparticle. Examples of nanoparticles that can be used to encapsulate one or more microRNAs (e.g., miR-603) and/or one or more anti-microRNAs (e.g., anti-miR-21) and can include a targeting moiety include, without limitation, liposomes, LNPs, extracellular vesicles, polymersomes, polymeric nanoparticles, and micelles.

A nanoparticle provided herein (e.g., a nanoparticle encapsulating one or more microRNAs such as miR-603 and/or one or more anti-microRNAs such as anti-miR-21 and including a targeting moiety) can encapsulate any appropriate microRNA(s). A microRNA can be any appropriate length. In some cases, a microRNA can be from about 21 to about 25 nucleotides in length. Examples of microRNAs that can encapsulated within a nanoparticle provided herein include, without limitation, miR-603, miR-34a, miR-218, miR-219, miR-183m, miR-451, miR-133, miR-134, miR-302c, miR-324, miR-379, miR-491, miR-340, miR-7, miR-128, miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, microRNAs in the miR-200 family, miR-203, miR-203b-3p, miR-205, miR-206, miR-217, miR-298, miR-375, miR-490-3p, miR-539, miR-542-3p, miR-613, miR-638, miR-940, and microRNAs in the let-7 family. When a microRNA is miR-603, the miR-603 can include, consist of, or consist essentially of the nucleotide sequence CACACACUGCAAUUACUUUUGC (SEQ ID NO:1). When a microRNA is miR-603, the miR-603 can include, consist of, or consist essentially of the nucleotide sequence AAAAGUAAUUGCAGUGUGUGUU (SEQ ID NO:2).

A miR-603 that consists essentially of the nucleotide sequence set forth in SEQ ID NO:1 or SEQ ID NO:2 is a nucleotide sequence that has zero, one, or two nucleotide substitutions within the articulated sequence of the sequence identifier (e.g., SEQ ID NO:1 or SEQ ID NO:2), has zero, one, two, three, four, or five nucleotides preceding the articulated sequence of the sequence identifier (e.g., SEQ ID NO:1 or SEQ ID NO:2), and/or has zero, one, two, three, four, or five nucleotides following the articulated sequence of the sequence identifier (e.g., SEQ ID NO:1 or SEQ ID NO:2), provided that miR-603 has the ability to reduce or eliminate expression of insulin-like growth factor-1 (IGF1) polypeptides and/or IGF1 receptor (IGF1R) polypeptides.

A nanoparticle provided herein (e.g., a nanoparticle encapsulating one or more microRNAs such as miR-603 and/or one or more anti-microRNAs such as anti-miR-21 and including a targeting moiety) can encapsulate any appropriate anti-microRNA(s). An anti-microRNA can be any appropriate length. In some cases, an anti-microRNA can include one or more locked nucleic acids (LNAs). In some cases, an anti-microRNA can be from about 17 to about 25 nucleotides in length (e.g., from about 17 to about 22 nucleotides in length, from about 17 to about 20 nucleotides in length, from about 20 to about 25 nucleotides in length, from about 22 to about 25 nucleotides in length, from about 18 to about 24 nucleotides in length, from about 20 to about 22 nucleotides in length, from about 18 to about 20 nucleotides in length, from about 19 to about 22 nucleotides in length, or from about 20 to about 23 nucleotides in length). Examples of anti-microRNAs that can encapsulated within a nanoparticle provided herein include, without limitation, anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b -5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, and anti-miR-1908. When an anti-microRNA is anti-miR-21, the anti-miR-21 can include, consist of, or consist essentially of the nucleotide sequence TCAACATCAGTCTG ATAAGCTA (SEQ ID NO:22).

An anti-miR-21 that consists essentially of the nucleotide sequence set forth in SEQ ID NO:22 is a nucleotide sequence that has zero, one, or two nucleotide substitutions within the articulated sequence of the sequence identifier (e.g., SEQ ID NO:22), has zero, one, two, three, four, or five nucleotides preceding the articulated sequence of the sequence identifier (e.g., SEQ ID NO:22), and/or has zero, one, two, three, four, or five nucleotides following the articulated sequence of the sequence identifier (e.g., SEQ ID NO:22), provided that the anti-miR-21 has the ability to reduce or eliminate cellular expression of miR-21.

In some cases, a microRNA that can be encapsulated within a nanoparticle provided herein (e.g., a nanoparticle encapsulating one or more microRNAs such as miR-603 and/or one or more anti-microRNAs such as anti-miR-21 and including a targeting moiety) can be complexed or mixed with one or more additional molecules. For example, a microRNA or an anti-microRNA that can be encapsulated within a nanoparticle provided herein can be complexed with one or more additional molecules that can increase endosomal escape of the microRNA or the anti-microRNA from the nanoparticle. In some cases, a microRNA or an anti-microRNA can be complexed with a polymer (e.g., a cationic polymer). Examples of polymers that can be complexed with a microRNA or an anti-microRNA that can be encapsulated within a nanoparticle provided herein include, without limitation PEI, poly-L-lysine, polyamine-based polymers, dextran, hyaluronic acid, and chitosan-based polymers. In some cases, a microRNA or an anti-microRNA can be mixed with an ionizable cationic amphiphilic molecule (e.g., an ionizable cationic lipid). Examples of ionizable cationic lipids that can be encapsulated within a nanoparticle provided herein include, without limitation SM-102, MC3, A6, DLin-MC3-DMA, A18-Iso5-2DC18, 9A1P9, L319, and LP000001. In some cases, a microRNA or an anti-microRNA can be complexed or mixed with one or more molecules described elsewhere (see, e.g., Han et al., Nat. Commun. 12:7233 (2021)).

When a microRNA or an anti-microRNA that can be encapsulated within a nanoparticle provided herein is complexed with PEI, the PEI can have any appropriate molecular weight (e.g., average molecular weight). In some cases, PEI can be linear. In some cases, PEI can be branched. For example, microRNA or anti-microRNA that can be encapsulated within a nanoparticle provided herein can be complexed with PEI having a molecular weight (e.g., an average molecule weight) of about 25 kDa.

In some cases, a microRNA or an anti-microRNA that can be encapsulated within a nanoparticle provided herein (e.g., a nanoparticle encapsulating one or more microRNAs such as miR-603 and/or one or more anti-microRNAs such as anti-miR-21 and including a targeting moiety) can be labelled. For example, a microRNA or an anti-microRNA can include a detectable label such that, after administering nanoparticles provided herein to a mammal, the location of the microRNA or the anti-microRNA within the mammal can be detected. In some cases, a detectable label can be radioactive. In some cases, a detectable label can be fluorescent. In some cases, a detectable label can be luminescent. In some cases, a detectable label can be a dye. Examples of detectable labels that can be included in a microRNA or an anti-microRNA that can be encapsulated within a nanoparticle provided herein include, without limitation, fluoresceins, Cy3, and Cy5.

A nanoparticle provided herein (e.g., a nanoparticle encapsulating one or more microRNAs such as miR-603 and/or one or more anti-microRNAs such as anti-miR-21 and including a targeting moiety) can include any appropriate targeting moiety. For example, a nanoparticle provided herein can be functionalized with any appropriate targeting moiety. A targeting moiety can be any type of molecule (e.g., nucleic acid or polypeptide such as an antibody). In some cases, a targeting moiety can be covalently attached to a nanoparticle provided herein. In some cases, a targeting moiety can be non-covalently attached to a nanoparticle provided herein. In some cases, a targeting moiety can be embedded within the lipid membrane (e.g., a lipid bilayer or a lipid layer) of a nanoparticle provided herein such that the targeting moiety is presented on the outside of the nanoparticle.

A targeting moiety that can be included in a nanoparticle provided herein (e.g., a nanoparticle encapsulating one or more microRNAs such as miR-603 and/or one or more anti-microRNAs such as anti-miR-21 and including a targeting moiety) can target any appropriate type of cell. In some cases, a targeting moiety that can be included in a nanoparticle provided herein can target (e.g., can target and internalize into) a cancer cell (e.g., a GBM cancer cell or a TNBC cell).

A targeting moiety that can be included in a nanoparticle provided herein (e.g., a nanoparticle encapsulating one or more microRNAs such as miR-603 and/or one or more anti-microRNAs such as anti-miR-21 and including a targeting moiety) can target any appropriate molecule. In some cases, a targeting moiety that can be included in a nanoparticle provided herein can target (e.g., can target and bind to) a polypeptide (e.g., a polypeptide receptor) presented on a cancer cell (e.g., a GBM cancer cell or a TNBC cell). For example, a targeting moiety that can be included in a nanoparticle provided herein can target (e.g., can target and bind to) a tumor-associated polypeptide (e.g., a cell surface tumor-associated polypeptide receptor). For example, a targeting moiety that can be included in a nanoparticle provided herein can target (e.g., can target and bind to) a tumor-specific polypeptide (e.g., a cell surface tumor-specific polypeptide receptor). Examples of polypeptides (e.g., polypeptide receptors) that can be targeted by a targeting moiety that can be included in a nanoparticle provided herein include, without limitation, integrin α5β1 polypeptides (e.g., integrin α5β1 polypeptides that are overexpressed on GBM cells and TNBC cells), epidermal growth factor receptor (EGFR) polypeptides (e.g., EGFR polypeptides that are overexpressed on GBM cells and TNBC cells), and transferrin receptor polypeptides (e.g., transferrin receptor polypeptides that are overexpressed on GBM cells and TNBC cells).

In some cases, a targeting moiety that can be included in a nanoparticle provided herein (e.g., a nanoparticle encapsulating one or more microRNAs such as miR-603 and/or one or more anti-microRNAs such as anti-miR-21 and including a targeting moiety) be a peptide mimetic. When a targeting moiety that can be included in a nanoparticle provided herein is a peptide mimetic, the targeting moiety can mimic the function of any appropriate polypeptide. For example, a targeting moiety that can be included in a nanoparticle provided herein can be a fibronectin-mimetic peptide.

In some cases, a targeting moiety that can be included in a nanoparticle provided herein (e.g., a nanoparticle encapsulating one or more microRNAs such as miR-603 and/or one or more anti-microRNAs such as anti-miR-21 and including a targeting moiety) can have any sequence. In some cases, a targeting moiety included in a nanoparticle provided herein can include a polypeptide having the amino acid sequence RGDSP (SEQ ID NO:3). In some cases, a targeting moiety included in a nanoparticle provided herein can include a polypeptide having the amino acid sequence RGDSP (SEQ ID NO:3) and can include a polypeptide having the amino acid sequence PHSRN (SEQ ID NO:4). In some cases, a targeting moiety included in a nanoparticle provided herein also can include one or more linkers. For example, a linker (e.g., a peptide linker) can separate the two polypeptides that can target (e.g., can target and bind to) a polypeptide (e.g., a polypeptide receptor). Examples of linkers that can be included in a targeting moiety included in a nanoparticle provided herein include, without limitation, SGSGSGSGSG (SEQ ID NO:17), SGSGSGSG (SEQ ID NO:18), GGGGGGGGGG (SEQ ID NO:19), and SSSSSSSSSS (SEQ ID NO:20). Examples of polypeptides that can be included in a targeting moiety that can be included in a nanoparticle provided herein include, without limitation, polypeptides including the amino acid sequence KSSPHSRNSGSGSGSGSGRGDSP (SEQ ID NO:5), polypeptides including the amino acid sequence PHSRNSGSGSGSGRGDSP (SEQ ID NO:6), polypeptides including the amino acid sequence KSSPHSRNGGGGGGGGGGRGDSP (SEQ ID NO:7), polypeptides including the amino acid sequence KSSPHSRNSSSSSSSSSSGRGDSP (SEQ ID NO:8), polypeptides including the amino acid sequence PHSRNSGSGSGSGSGRGDSP (SEQ ID NO:9), polypeptides including the amino acid sequence KSSSSSPHSRNSGSGSGSGSGRGDSP (SEQ ID NO:10), RGD, and GRGDSP (SEQ ID NO:21).

In some cases, a targeting moiety included in a nanoparticle provided herein (e.g., a nanoparticle encapsulating one or more microRNAs such as miR-603 and/or one or more anti-microRNAs such as anti-miR-21 and including a targeting moiety) can include a polypeptide that comprises, consists of, or consists essentially of the amino acid sequence KSSPHSRNSGSGSGSGSGRGDSP (SEQ ID NO:5). A targeting moiety that consists essentially of the amino acid sequence set forth in SEQ ID NO:5 is an amino sequence that has zero, one, or two amino acid residue substitutions within the articulated sequence of the sequence identifier (e.g., SEQ ID NO:5), has zero, one, two, three, four, or five amino acid residues preceding the articulated sequence of the sequence identifier (e.g., SEQ ID NO:5), and/or has zero, one, two, three, four, or five amino acid residues following the articulated sequence of the sequence identifier (e.g., SEQ ID NO:5), provided that amino acid sequence has the ability to bind integrin α5β1 polypeptides.

Any appropriate method can be used to incorporate a targeting moiety into a nanoparticle provide herein (e.g., a nanoparticle encapsulating one or more microRNAs such as miR-603 and/or one or more anti-microRNAs such as anti-miR-21 and including a targeting moiety). In some cases, a targeting moiety can be synthesized as an amphiphile and incorporated into the lipid membrane (e.g., a lipid bilayer or a lipid layer) of a nanoparticle. For example, a targeting moiety can be conjugated to a hydrophobic tail and incorporated into the lipid membrane (e.g., a lipid bilayer or a lipid layer) of a nanoparticle.

In some cases, a targeting moiety included in a nanoparticle provided herein (e.g., a nanoparticle encapsulating one or more microRNAs such as miR-603 and/or one or more anti-microRNAs such as anti-miR-21 and including a targeting moiety) can be conjugated to a hydrophobic tail having the structure (Cₙ)₁₋₃—R—, where n can be 6-22, and where R is connector that can be an amino acid (e.g., a glutamic acid (Glu) or an aspartic acid (Asp)) or an alkyl chain having from 2-10 carbon atoms (C₂₋₁₀). For example, a hydrophobic tail can include one, two, or three alkyl chains. In some cases, an alkyl chain of a hydrophobic tail that can be conjugated to a targeting moiety included in a nanoparticle provided herein can be a saturated alkyl chain. In some cases, an alkyl chain of a hydrophobic tail that can be conjugated to a targeting moiety included in a nanoparticle provided herein can be an unsaturated alkyl chain. An alkyl chain of a hydrophobic tail that can be conjugated to a targeting moiety included in a nanoparticle provided herein can include a hydrocarbon chain having any number of carbon molecules. For example, an alkyl chain of a hydrophobic tail that can be conjugated to a targeting moiety included in a nanoparticle provided herein can be a hydrocarbon chain having from about C₆ to about C₂₂. For example, an alkyl chain of a hydrophobic tail that can be conjugated to a targeting moiety included in a nanoparticle provided herein can be a C₁₆ hydrocarbon chain.

In some cases, a targeting moiety can be conjugated directly to a hydrophobic tail.

In some cases, one or more spacers can be present in between a targeting moiety and a hydrophobic tail. When a spacer separates the targeting moiety and the hydrophobic tail, the spacer can be any appropriate spacer. In some cases, a spacer can be a hydrophobic spacer. In some cases, a spacer can be a carbon chain. In some cases, a spacer can be a peptide spacer. Examples of spacers that can be present in between a targeting moiety conjugated to a hydrophobic tail that can be included in a nanoparticle provided herein include, without limitation, C_{y} where y can be 2-24, polyethylene glycol (PEG), and polyethylene oxide (PEO).

In some cases, a targeting moiety conjugated to a hydrophobic tail that can be included in a nanoparticle provided herein (e.g., a nanoparticle encapsulating one or more microRNAs such as miR-603 and/or one or more anti-microRNAs such as anti-miR-21 and including a targeting moiety) can include the structure (C₁₆)₂-Glu-C₂-KSSPHSRNSGSGSGSGSGRGDSP (SEQ ID NO:5).

In some cases, a targeting moiety conjugated to a hydrophobic tail that can be included in a nanoparticle provided herein (e.g., a nanoparticle encapsulating one or more microRNAs such as miR-603 and/or one or more anti-microRNAs such as anti-miR-21 and including a targeting moiety) can be as described elsewhere (see, e.g., U.S. Pat. No. 8,343,539 (see, e.g., Table 1 and Table 2), and Kuang et al., Adv. Drug Deliv. Rev., 110-111:80-101 (2017)).

A nanoparticle provided herein (e.g., a nanoparticle encapsulating one or more microRNAs such as miR-603 and/or one or more anti-microRNAs such as anti-miR-21 and including a targeting moiety) can include any amount of a targeting moiety. In some cases, a nanoparticle provided herein can include from about 2 mol % to about 20 mol % (e.g., from about 2 mol % to about 18 mol %, from about 2 mol % to about 15 mol %, from about 2 mol % to about 12 mol %, from about 2 mol % to about 10 mol %, from about 2 mol % to about 7 mol %, from about 5 mol % to about 20 mol %, from about 8 mol % to about 20 mol %, from about 10 mol % to about 20 mol %, from about 12 mol % to about 20 mol %, from about 15 mol % to about 20 mol %, from about 17 mol % to about 20 mol %, from about 5 mol % to about 15 mol %, from about 8 mol % to about 10 mol %, from about 5 mol % to about 8 mol %, from about 7 mol % to about 10 mol %, from about 10 mol % to about 15 mol %, or from about 12 mol % to about 18 mol %) of a targeting moiety. For example, a nanoparticle provided herein can include from about 3.5 mol % of a targeting moiety. For example, a nanoparticle provided herein can include from about 5 mol % of a targeting moiety.

A nanoparticle provided herein (e.g., a nanoparticle encapsulating one or more microRNAs such as miR-603 and/or one or more anti-microRNAs such as anti-miR-21 and including a targeting moiety) can be any size (e.g., can have any longest dimension such as a diameter). In some cases, a nanoparticle provided herein can have a longest dimension (e.g., a diameter) of from 50 nm to about 500 nm (e.g., from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, from 50 nm to about 500 nm, or from 50 nm to about 500 nm). For example, a nanoparticle provided herein can have a longest dimension (e.g., a diameter) of from about 100 nm to about 200 nm (e.g., about 150 nm).

A nanoparticle provided herein (e.g., a nanoparticle encapsulating one or more microRNAs such as miR-603 and/or one or more anti-microRNAs such as anti-miR-21 and including a targeting moiety) can include one or more additional molecules. In some cases, an additional molecule that can be included in a nanoparticle provided herein can increase the stability (e.g., the colloidal stability) of the nanoparticle. In some cases, an additional molecule that can be included in a nanoparticle provided herein can reduce or prevent aggregation between multiple nanoparticles provided herein. Examples of molecules that can be included in a nanoparticle provided herein include, without limitation, PEG (e.g., PEG750, PEG2000, and PEG5000) and PEO.

Any appropriate method can be used to make a nanoparticle provided herein (e.g., a nanoparticle encapsulating one or more microRNAs such as miR-603 and/or one or more anti-microRNAs such as anti-miR-21 and including a targeting moiety). For example, a lipid film technique (e.g., a dry lipid film technique) or a microfluidic device can be used to make a nanoparticle provided herein.

In some cases, nanoparticles provided herein (e.g., nanoparticles encapsulating one or more microRNAs such as miR-603 and/or one or more anti-microRNAs such as anti-miR-21 and including a targeting moiety) can be formulated into a composition (e.g., a pharmaceutically acceptable composition). For example, a composition including nanoparticles provided herein can include one or more pharmaceutically acceptable carriers (additives), excipients, and/or diluents. Examples of pharmaceutically acceptable carriers, excipients, and diluents that can be used in a composition described herein include, without limitation, PEG, PEO, sterols, stanols, cholesterol, β-sitosterol, phosphate-buffered saline, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum polypeptides (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A pharmaceutical composition can be formulated for local administration or systemic administration.

A pharmaceutical composition can be formulated for administration in solid or liquid form including, without limitation, sterile solutions, suspensions, sustained-release formulations, tablets, capsules, pills, powders, and granules.

In some cases, a composition including nanoparticles provided herein (e.g., nanoparticles encapsulating one or more microRNAs such as miR-603 and/or one or more anti-microRNAs such as anti-miR-21 and including a targeting moiety) can be formulated as a delivery system. For example, a composition including nanoparticles provided herein can be formulated as a controlled-release delivery system for the one or more microRNAs and/or the one or more anti-microRNAs encapsulated within the nanoparticles. Examples of types of controlled-release delivery that a composition including nanoparticles described herein can be formulated to include, without limitation, induced release, burst release, slow release, delayed release, and sustained release.

This document also provides methods and materials for using nanoparticles provided herein (e.g., nanoparticles encapsulating one or more microRNAs such as miR-603 and/or one or more anti-microRNAs such as anti-miR-21 and including a targeting moiety). In some cases, a mammal (e.g., a human) having cancer (e.g., a CNS cancer such as a GBM or a breast cancer such as a TNBC) can be treated by administering nanoparticles provided herein (e.g., a composition including nanoparticles provided herein) to the mammal.

Any type of mammal having cancer (e.g., a CNS cancer such as a GBM or a breast cancer such as a TNBC) can be treated using the methods and materials described herein (e.g., by administering nanoparticles provided herein). Examples of mammals that can be treated as described herein include, without limitation, humans, non-human primates such as monkeys, dogs, cats, horses, cows, pigs, sheep, rabbits, mice, and rats. In some cases, a human having cancer (e.g., a CNS cancer such as a GBM or a breast cancer such as a TNBC) can be treated by administering nanoparticles provided herein (e.g., nanoparticles encapsulating one or more microRNAs such as miR-603 and/or one or more anti-microRNAs such as anti-miR-21 and including a targeting moiety).

In some cases, the methods described herein can include identifying a mammal (e.g., a human) as having cancer (e.g., a CNS cancer such as a GBM or a breast cancer such as a TNBC). Any appropriate method can be used to identify a mammal as having cancer (e.g., a glioma such as a GBM or a breast cancer such as a TNBC). For example, medical history (e.g., a history of having had a prior CNS cancer), neurological examinations (e.g., to check vision, hearing, balance, coordination, strength, and/or reflexes), physical examinations such as breast examinations (e.g., to identify the presence of a breast lump, change in size and/or shape of the breast, changes to the skin of breast, and/or an inverted nipple and/or redness or pitting of skin over breast), imaging techniques such as magnetic resonance imaging (MRI), mammograms, magnetic resonance spectroscopy, computed tomography (CT) scanning, and positron emission tomography (PET) scanning (e.g., to determine the location and size of a brain tumor), and/or biopsy techniques can be used to identify mammals (e.g., humans) having, or at risk of developing, a cancer (e.g., a CNS cancer such as a GBM or a breast cancer such as a TNBC).

A mammal (e.g., a human) having any type of cancer can be treated as described herein (e.g., by administering nanoparticles provided herein). In some cases, a cancer can be a blood cancer (e.g., lymphomas and leukemias). In some cases, a cancer can include one or more solid tumors. In some cases, a cancer can be a primary cancer. In some cases, a cancer can be a metastatic cancer. In some cases, a cancer can be a recurrent cancer. In some cases, a cancer can be a chemotherapy-resistant cancer. In some cases, a cancer can be a radiation therapy-resistant cancer. In some cases, a cancer can be a CNS cancer. In some cases, a cancer can be a breast cancer. Examples of cancers that can be treated as described herein include, without limitation, gliomas (e.g., brain stem gliomas, GBMs, and diffuse intrinsic pontine gliomas), astrocytomas, oligodendrogliomas, oligoastrocytomas, ependymomas, medulloblastomas, meningiomas, breast cancers (e.g., TNBCs), colon cancers, liver cancer, pancreatic cancer, and prostate cancer.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal in need thereof (e.g., a mammal having cancer such as a human having a CNS cancer such as a GBM or a breast cancer such as a TNBC) to reduce or eliminate the number of cancer cells present within a mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal in need thereof (e.g., a mammal having cancer such as a human having a CNS cancer such as a GBM or a breast cancer such as a TNBC) to improve survival of the mammal. For example, disease-free survival (e.g., recurrence-free survival) can be improved using the materials and methods described herein. For example, progression-free survival can be improved using the materials and methods described herein. In some cases, the materials and methods described herein can be used to improve the survival of a mammal having cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal in need thereof (e.g., a mammal having cancer such as a human having a CNS cancer such as a GBM or a breast cancer such as a TNBC) to reduce or eliminate expression of IGF1 polypeptides and/or IGF1R polypeptides in cancer cells within the mammal. Any appropriate method can be used to determine the presence, absence, or level of expression of IGF1 polypeptides and/or IGF1R polypeptides in cancer cells within the mammal. In some cases, the materials and methods described herein can be used to reduce expression of IGF1 polypeptides and/or IGF1R polypeptides in cancer cells within a mammal having cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal in need thereof (e.g., a mammal having cancer such as a human having a CNS cancer such as a GBM or a breast cancer such as a TNBC) to reduce stemness of cancer cells within the mammal. Any appropriate method can be used to determine stemness of cancer cells. In some cases, the formation of spheres (e.g., cellular spheres such as neurospheres) can be used to determine the stemness of cancer cells. In some cases, the presence, absence, or level of expression of one or more stem cell markers can be used to determine the stemness of cancer cells. In some cases, the materials and methods described herein can be used to reduce the stemness of cancer cells within a mammal having cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal in need thereof (e.g., a mammal having cancer such as a human having a CNS cancer such as a GBM or a breast cancer such as a TNBC) to decrease proliferation of cancer cells within the mammal. Any appropriate method can be used to determine proliferation of cancer cells. In some cases, the presence, absence, or level of expression of one or more proliferation markers (e.g., Ki67 and ATP) can be used to determine proliferation of cancer cells. In some cases, the materials and methods described herein can be used to decrease proliferation of cancer cells within a mammal having cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal having been previously treated and/or scheduled to receive treatment for a cancer (e.g., a CNS cancer such as a GBM or a breast cancer such as a TNBC) to delay or prevent recurrence of the cancer in the mammal. For example, tumor initiation can be delayed or prevented using the materials and methods described herein. In some cases, the materials and methods described herein can be used to delay the recurrence of the cancer in a mammal having been previously treated and/or scheduled to receive treatment for a cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal having been previously treated and/or scheduled to receive treatment for a cancer (e.g., a CNS cancer such as a GBM or a breast cancer such as a TNBC) to increase the sensitivity of the cancer to the treatment. For example, the materials and methods described herein can be used to increase the sensitivity of cancer cells within a mammal having been previously treated and/or scheduled to receive treatment for a cancer (e.g., a CNS cancer such as a GBM or a breast cancer such as a TNBC) to a radiation therapy (e.g., IR therapy). In some cases, the materials and methods described herein can be used to increase the sensitivity of cancer cells within a mammal having been previously treated and/or scheduled to receive treatment for a cancer (e.g., a CNS cancer such as a GBM or a breast cancer such as a TNBC) by, for example, 10, 20, 30, 40, 50, 60, 70, 80, 90, 95, or more percent.

In some cases, nanoparticles provided herein (e.g., nanoparticles encapsulating one or more microRNAs such as miR-603 and/or one or more anti-microRNAs such as anti-miR-21 and including a targeting moiety), when administered to a mammal (e.g., a human), can cross the blood brain barrier. For example, a composition including nanoparticles provided herein, when administered to a mammal (e.g., a human), can cross the blood brain barrier and enter the brain of that mammal thereby delivering the nanoparticles provided herein to the brain of that mammal.

Nanoparticles provided herein (e.g., nanoparticles encapsulating one or more microRNAs such as miR-603 and/or one or more anti-microRNAs such as anti-miR-21 and including a targeting moiety) can be administered to a mammal (e.g., a human) having cancer (e.g., a CNS cancer such as GBM or a breast cancer such as a TNBC) in any appropriate amount (e.g., any appropriate dose). For example, an effective amount of a composition containing nanoparticles provided herein can be any amount that can treat a mammal having cancer as described herein without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer may require an increase or decrease in the actual effective amount administered.

Nanoparticles provided herein (e.g., nanoparticles encapsulating one or more microRNAs such as miR-603 and/or one or more anti-microRNAs such as anti-miR-21 and including a targeting moiety) can be administered to a mammal (e.g., a human) having cancer (e.g., a CNS cancer such as GBM or a breast cancer such as a TNBC) in any appropriate frequency. The frequency of administration can be any frequency that can treat a mammal having cancer without producing significant toxicity to the mammal. For example, the frequency of administration can be from about once a day to about once a week, from about once a week to about once every two weeks, or from about once a week to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

Nanoparticles provided herein (e.g., nanoparticles encapsulating one or more microRNAs such as miR-603 and/or one or more anti-microRNAs such as anti-miR-21 and including a targeting moiety) can be administered to a mammal (e.g., a human) having cancer (e.g., a CNS cancer such as GBM or a breast cancer such as a TNBC) for any appropriate duration. An effective duration for administering or using a composition containing nanoparticles provided herein can be any duration that can treat a mammal having cancer without producing significant toxicity to the mammal. For example, the effective duration can vary from several days to several weeks, from several weeks to several months, or from several months to a year. Multiple factors can influence the actual effective duration used for a particular treatment. For example, an effective duration can vary with the frequency of administration, effective amount, use of multiple treatment agents, and/or route of administration.

In some cases, nanoparticles provided herein (e.g., nanoparticles encapsulating one or more microRNAs such as miR-603 and/or one or more anti-microRNAs such as anti-miR-21 and including a targeting moiety) can be administered to a mammal in need thereof (e.g., a mammal having cancer such as a human having a CNS cancer such as a GBM or a breast cancer such as a TNBC) as the sole active ingredient used to treat the mammal. For example, a composition containing nanoparticles provided herein can include the nanoparticles as the sole active ingredient in the composition that is effective to treat a mammal having cancer (e.g., a CNS cancer such as GBM or a breast cancer such as a TNBC).

In some cases, methods for treating a mammal (e.g., a human) having cancer (e.g., a CNS cancer such as GBM or a breast cancer such as a TNBC) as described herein (e.g., by administering nanoparticles provided herein) can include administering to the mammal nanoparticles provided herein (e.g., nanoparticles encapsulating one or more microRNAs such as miR-603 and/or one or more anti-microRNAs such as anti-miR-21 and including a targeting moiety) together with one or more (e.g., one, two, three, four, five or more) additional anti-cancer agents (e.g., chemotherapeutic agents, immunotherapies, and gene therapies) used to treat cancer. Examples of anti-cancer agents that can be administered together with nanostructures provided herein include, without limitation, doxorubicin, temozolomide, cisplatin, 5FU, paclitaxel, gemcitabine, cyclophosphamide, and any combinations thereof. In cases where nanoparticles provided herein are used in combination with additional agents used to treat cancer, the one or more additional agents can be administered at the same time (e.g., in a single composition containing both nanoparticles provided herein and the one or more additional agents) or independently. For example, nanoparticles provided herein can be administered first, and the one or more additional agents administered second, or vice versa.

In some cases, methods for treating a mammal (e.g., a human) having cancer (e.g., a CNS cancer such as GBM or a breast cancer such as a TNBC) as described herein (e.g., by administering nanoparticles provided herein) can include administering to the mammal nanoparticles provided herein (e.g., nanoparticles encapsulating one or more microRNAs such as miR-603 and/or one or more anti-microRNAs such as anti-miR-21 and including a targeting moiety) together with one or more (e.g., one, two, three, four, five or more) additional therapies used to treat cancer. Examples of therapies that can be used to treat cancer include, without limitation, surgery and radiation therapy (e.g., IR therapy). In cases where nanoparticles provided herein are used in combination with one or more additional therapies used to treat cancer, the one or more additional therapies can be performed at the same time or independently of the administration of the nanoparticles provided herein. For example, the nanoparticles provided herein can be administered before, during, or after the one or more additional therapies are performed.

The invention will be further described in the following examples, which do not limit the scope of the invention described in the claims.

### EXAMPLES

### Example 1: Targeted Liposomes Encapsulating MiR-603 Complexes Enhance Radiation Sensitivity of Patient-Derived Glioblastoma Stem-Like Cells

This Example describes the design and generation of targeted liposomes that can be used to treat GBM. For example, liposomes encapsulating miR-603 and containing a targeting moiety that can specifically target (e.g., target and bind) the integrin α5β1 polypeptide that is overexpressed on GBM cells, such as the PR_b peptide-amphiphile, can be internalized into GBM cells and can deliver miR-603 to the GBM cells.

### Materials and Methods

### Materials and Cells

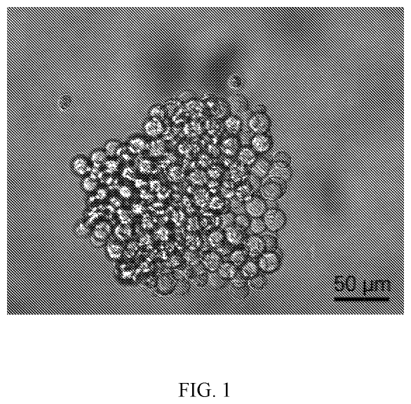

miR-603 was purchased from GE Healthcare Dharmacon, branched polyethylenimine (PEI) 25 kDa, sephadex G-50 and calcein were purchased from Sigma-Aldrich. Dipalmitoylphosphatidylcholine (DPPC), cholesterol and 1,2-dipalmitoyl-sn-glycero-3-phosphoethanolamine-N-[methoxy(polyethylene glycol)-750] (DPPE-PEG750) (ammonium salt) were purchased from Avanti Polar Lipids. Dialysis membrane 1000 kDa MWCO was purchased from Spectrum Laboratories. GBM-CCC-001, GBM-CCC-002, and GBM-CCC-003GBM are patient-derived stem-like cells. The cells were passaged as neurospheres in Dulbecco's modified Eagle's medium/F-12 supplemented with 20 ng/ml recombinant human (rh) EGF, 10 ng/mL rh bFGF, 2 μg/mL heparin, B-27 supplement, 100 U/mL penicillin, and 100 ng/mL streptomycin in ultra-low attachment flasks at 37° C. incubator with 5% CO₂. A representative image is shown in FIG. 1.

### Preparation and Characterization of MiR-603 PEI Complexes

Fluorescently labeled miR-603 (active strand: 5′-P-CACACACUGCAAUUACUUUUGC-3′ (SEQ ID NO:1), passenger strand: 5′-fluorescein-AAAAGUAAUUGCAGUGUGUGUU-3′ (SEQ ID NO:2)) was complexed using branched PEI 25 kDa. 200 nM miR-603 dissolved in 6 mM HEPES buffer (pH 7.4) was mixed with an equal volume of PEI in 6 mM HEPES buffer (pH 7.4) at different nitrogen to phosphate (N:P) ratio to yield a final miR-603 concentration of 100 nM. The mixture was vortexed for 5 seconds followed by 30 minutes incubation period at room temperature. The size and zeta potential of the miR-603/PEI complexes were measured using a Zetasizer (Malvern Panalytical).

### Preparation and Characterization of Liposomes

Liposomes were prepared using a dry lipid film technique. Stock solutions of DPPC, cholesterol and DPPE-PEG750 in chloroform were mixed in the following molar ratio 64:35: 1 respectively, to give a total lipid concentration of 10 mM. PR_b-amphiphiles having the structure (C₁₆)₂-Glu-C₂-KSSPHSRNSGSGSGSGSGRGDSP (SEQ ID NO:5) were used. To prepare integrin targeted liposomes, the PR_b-amphiphile was synthesized as described elsewhere (see, e.g., Atchison et al., Langmuir, 26:14081-14088 (2010); Adil et al., Langmuir, 30:3802-3810 (2014)) and was mixed with the other liposomal components at an initial concentration of 5 mol % (DPPC:cholesterol:DPPE-PEG750:PR_b at 59:35:1:5 mol %). The lipid mixture was placed in a rotary evaporator flask at 50° C. to remove the organic solvent, followed by further drying the sample under vacuum overnight to generate a uniform dry lipid film. To prepare calcein loaded liposomes, the lipid film was hydrated with 2 mM calcein at 45° C. for 2 hours. The generated multilamellar vesicles were subjected to extrusion through 200 nm polycarbonate membrane for 21 cycles at 45° C. The unencapsulated dye was removed by gel permeation chromatography using Sephadex G-50 prepacked column. To prepare miR-603 or miR-603/PEI loaded liposomes, the lipid film was hydrated with 1 mL of either 100 nM miR-603 or miR-603/PEI complex solution in 6 mM HEPES buffer pH 7.4 at 45° C. for 2 hours. The generated multilamellar vesicles were extruded as mentioned above and the unencapsulated miR-603 or miR-603/PEI complexes were removed through overnight dialysis at 4° C. using a 1000 kDa MWCO dialysis membrane. The size and zeta potential of all generated liposomes were measured using a Zetasizer (Malvern Panalytical). The final peptide concentration on the surface of the liposomes was evaluated using a BCA protein assay (Thermo Fisher Scientific) following the manufacturer's protocol. The encapsulation efficiency of miR-603 was determined by fluorescence spectroscopy using a BioTek Synergy H1 microplate reader.

### Cryogenic Transmission Electron Microscopy (Cryo-TEM)

The morphology of miR-603/PEI complex at N:P ratio of 6:1 and miR-603/PEI loaded liposomes was examined with cryo-TEM. Briefly, 5 μL of an aqueous suspension of either miR-603/PEI or liposomes encapsulating the complexes were deposited onto carbon copper grids (Ted Pella) pretreated with glow discharge for 40 seconds and vitrified in liquid ethane by Vitroblot using the following parameters (4 second blot time, 0 offset, 0 second wait time, 0 second drain time, 100% relative humidity). The prepared sample grids were stored under liquid nitrogen until they were transferred to a FEI Tecnai-12 TEM operated with an acceleration voltage of 100 keV (Integrated Imaging Center, Institute for NanoBioTechnology). Images were acquired using an Eagle 2k CCD camera and SIS Megaview III wide-angle CCD camera.

### Expression of Integrin α₅β₁

The cell expression of integrin α₅β₁ in GSC neurospheres was verified. GBM-CCC-001, GBM-CCC-002 and GBM-CCC-003 neurospheres were collected into 15 mL Falcon tubes, pelleted down, and dissociated into single cells. About 200,000 cells were transferred into Eppendorf tubes and incubated with the primary anti-integrin α₅β₁ antibody MAB1969 (Millipore) or mouse IgG isotype control (Sigma-Aldrich) at 1:100 dilution in 1% w/v bovine serum albumin (BSA) in phosphate buffer saline (PBS), PBSA, at 4° C. for 30 minutes. Following the incubation period, cells were pelleted and washed twice with ice-cold PBSA, then incubated with the FITC-conjugated anti-mouse IgG secondary antibody (Thermo Fisher Scientific) for 30 minutes at 4° C. Finally, cells were pelleted and washed twice with ice-cold PBSA, and flow cytometric analysis was performed immediately using BD FACSCanto (Integrated Imaging Center, Institute for NanoBioTechnology). Cell autofluorescence was subtracted from all measurements.

### Flow Cytometry

GSC neurospheres (GBM-CCC-001, GBM-CCC-002 and GBM-CCC-003) were collected into 15 mL Falcon tubes, pelleted down, and dissociated into single cells. About 200,000 cells were transferred into Eppendorf tubes in neurosphere culture media and treated with 2 mM calcein loaded PR_b liposomes or non-targeted liposomes at a lipid concentration of 150 μM for 48 hours at 37° C. on a shaker. After the incubation period, cells were pelleted down and washed twice with PBS. Flow cytometric analysis was carried out immediately using BD FACSCanto. Non-treated cells that received only media served as a control. Cell autofluorescence was subtracted from all measurements. For the blocking experiment, the same procedure was followed, except cells were preincubated with 1 mg/mL free PR_b peptide in media for 1 hour at 37° C. prior to addition of the PR_b liposomes and incubation for an additional 1 hour at 37° C.

### Confocal Microscopy

GBM neurospheres (GBM-CCC-001, GBM-CCC-002 and GBM-CCC-003) were collected into 15 mL Falcon tubes, pelleted down, and dissociated into single cells. About 200,000 cells were transferred into Eppendorf tubes in neurosphere culture media and treated with 2 mM calcein loaded PR_b liposomes or non-targeted liposomes at a lipid concentration of 150 μM for 48 hours at 37° C. on a shaker. Cells were then pelleted down, washed twice with PBS, and mounted onto coverslips in a 12 well plate via centrifugation at 300 g for 10 minutes. Cells were fixed using 4% paraformaldehyde solution in PBS for 30 minutes. Nuclear staining was carried out using the cell membrane permeable dye Hoechst 33342

(Thermo Fisher Scientific) at a concentration of 2.0 μmol/mL, and the cell membrane was stained with cell impermeable AlexaFluor647 wheat germ agglutinin (Thermo Fisher Scientific) at 5.0 μg/mL in PBS for 15 minutes. Cells were mounted onto glass slides using Prolong Gold and imaged with a Carl Zeiss LSM700 confocal microscope (Integrated Imaging, Institute for NanoBioTechnology).

### RNA Extraction and Quantitative Real-Time PCR (qPCR)

GBM-CCC-001 neurospheres were pelleted down and dissociated into single cells. 200,000 cells were seeded in Eppendorf tubes and incubated with either PBS (control), PR_b empty liposomes, or PR_b liposomes encapsulating miR-603 or miR-603/PEI complexes (final working concentration of miR-603 was 100 nM) for 48 hours on a shaker at 37° C. Total RNA was isolated from cells using miRNeasy mini kit (Qiagen, 217004). cDNA synthesis was performed using the miScript II RT kit (Qiagen, 218160) according to manufacturer's protocol. mRNA transcripts were quantified using SYBR Green (Bio-Rad) on the Bio-Rad CFX96 Real-Time PCR Detection System. The following qPCR primers were used:

Hs_miR-603_3 miScript Primer (Qiagen, MS00037933),
IGF1:
(forward primer; SEQ ID NO: 11)
5′-GCAGCACTCATCCACGATGC-3′,
(reverse primer; SEQ ID NO: 12)
5′-TGTGGAGACAGGGGCTTTTATTTC-3′.
IGF1R:
(forward primer; SEQ ID NO: 13)
5′-AAGTTCTGGTTGTCGAGGA-3′,
(reverse primer; SEQ ID NO: 14)
5′-GAGCAGCTAGAAGGGAATTAC-3′.
18s:
(forward primer; SEQ ID NO: 15)
5′-TTGCCCTCCAATGGATCCT-3′,
(reverse primer; SEQ ID NO:16)
5′-GGGAGGTAGTGACGAAAAATAACAAT-3′.

### Limiting Dilution Assay

GBM-CCC-001 neurospheres were pelleted down, and dissociated into single cells. 200,000 cells were seeded in Eppendorf tubes and incubated with either PBS (control), PR_b empty liposomes, or PR_b liposomes encapsulating miR-603 or miR-603/PEI complexes (final working concentration of miR-603 was 100 nM) for 24 or 48 hours on a shaker at 37° C. After incubation, cells were irradiated with X-rays (6 Gy) and briefly washed with PBS. Single-cell suspensions of GBM-CCC-001 cells were then prepared, serially diluted, and plated into 96-well ultra-low attachment plates containing 100 μL neurosphere culture media at various seeding densities. After two weeks each well was scored for the absence or presence of tumor spheres by visual inspection (at least one aggregate of ≥50 cells). Data were analyzed using Extreme Limiting Dilution Analysis (ELDA, bioinf.wehi.edu.au/software/elda/).

### Results and Discussion

### Encapsulation of MiR-603 PEI Complexes into PR_b Liposomes

Anionic miR-603 was complexed with the cationic branched PEI at different nitrogen to phosphate (N:P) ratios. Dynamic light scattering (DLS) was used to measure the hydrodynamic diameter of the complexes, and zeta potential was evaluated via electrophoretic light scattering. The results are shown in Table 1. The zeta potential increased from −19 to 20 mV on average as the N:P ratio increased. Complexes at N:P ratio of 6 were used for further experiments as DLS showed only one peak with the smallest polydispersity index (PDI) of 0.11±0.04, and it was shown before that siRNA/PEI complexes at this ratio were more efficient at mRNA silencing compared to other ratios delivered to cells either free in solution or encapsulated in peptide functionalized liposomes. At N:P=6, the hydrodynamic diameter of the miR-603/PEI complexes was 103.9±24.4 nm and the zeta potential was 20.2±1.1 mV.

TABLE 1
Characterization of miR-603/PEI Complexes
at Different N:P Ratios.

| N:P ratio^{a} | diameter (nm)^{b} | PDI^{c} | zeta potential (mV) |
|---|---|---|---|
| 2 | 185.8 ± 73.9 | 0.56 ± 0.18 | −18.9 ± 2.0 |
| 4 | 103.1 ± 30.2 | 0.43 ± 0.49 | 9.0 ± 1.8 |
| 6 | 103.9 ± 24.4 | 0.11 ± 0.04 | 20.2 ± 1.1 |
| 8 | 99.0 ± 16.6 | 0.26 ± 0.04 | 18.9 ± 1.6 |

^{a}Data are reported as mean + SD (n = 3).
^{b}Hydrodynamic diameters are reported based on intensity from DLS.
^{c}Polydispersity index.

Targeted and non-targeted liposomes were prepared encapsulating miR-603 or miR-603/PEI complexes and characterized in term of size, zeta potential and encapsulation efficiency (Table 2). All liposomes used in this study had 1 mol % DPPE-PEG750 to prevent aggregation of the nanoparticles. The peptide concentration on the surface of the PR_b-functionalized liposomes was 3.1±0.5 mol %. The size of the liposomes was similar for all formulations tested. The presence of the positively charged PR_b peptide on the surface of the nanoparticles had an effect on the zeta potential of the liposomes, as it went from −25 mV for non-targeted formulations to about 5 mV for the PR_b-functionalized liposomes. The encapsulation efficiency of uncomplexed miR-603 was about 70% for both targeted and non-targeted formulations, and it decreased when miR-603 was encapsulated as complexes with PEI.

TABLE 2
Characterization of Liposomes Encapsulating
miR-603 or miR-603/PEI Complexes at N:P = 6

| | encapsulating | diameter | | zeta potential | encapsulation |
|---|---|---|---|---|---|
| formulation^{a} | load | (nm)^{b} | PDI^{c} | (mV) | efficiency (%) |
| non-targeted | miR-603 | 174.5 ± 2.0 | 0.025 ± 0.006 | −25.8 ± 0.5 | 70 ± 4 |
| liposomes | | | | | |
| non-targeted | miR-603/PEI | 173.6 ± 0.7 | 0.068 ± 0.008 | −24.7 ± 2.2 | 44 ± 5 |
| liposomes | | | | | |
| PR_b liposomes | miR-603 | 176.8 ± 1.5 | 0.029 ± 0.002 | 6.3 ± 0.1 | 69 ± 7 |
| PR_b liposomes | miR-603/PEI | 173.2 ± 3.8 | 0.036 ± 0.030 | 4.0 ± 1.3 | 58 ± 3 |

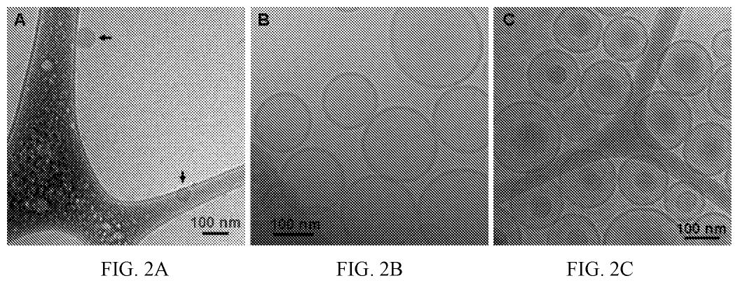

PR_b liposomes encapsulating miR-603/PEI complexes at N:P=6 were visualized via cryo-TEM (FIG. 2). For comparison empty PR_b liposomes and free complexes are also shown. Cryo-TEM images verified the presence of encapsulated miR-603/PEI complexes in unilamellar liposomes. The complexes had a diameter of 68±14 nm, the empty PR_b liposomes were 136±34 nm, and the diameter of the liposomes with the encapsulated complexes was 141±34. There was no significant statistical difference between the diameters of the two liposomal formulations. Differences between DLS and cryo-TEM measurements can be attributed to the fact that DLS measures the hydrodynamic diameter, and that in polydisperse samples the presence of bigger particles shifts DLS measurements towards larger values.

### Targeting Patient-Derived Glioblastoma Cell Lines with PR_b Liposomes

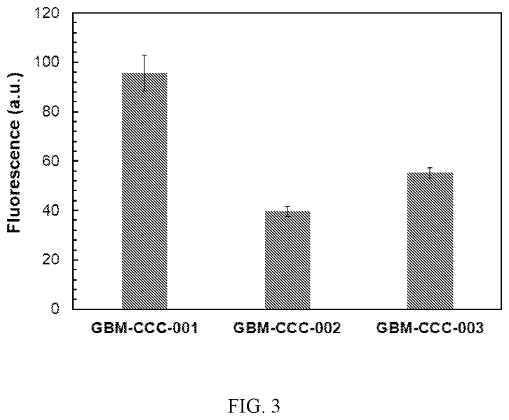

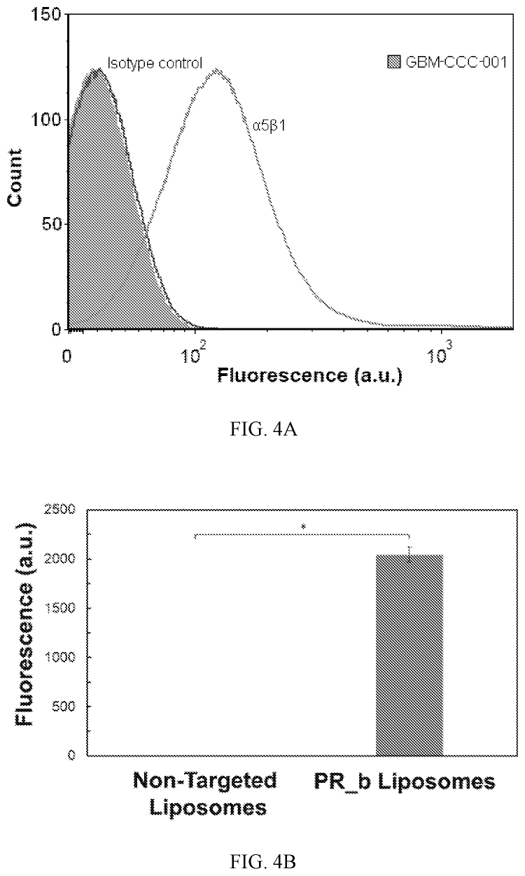

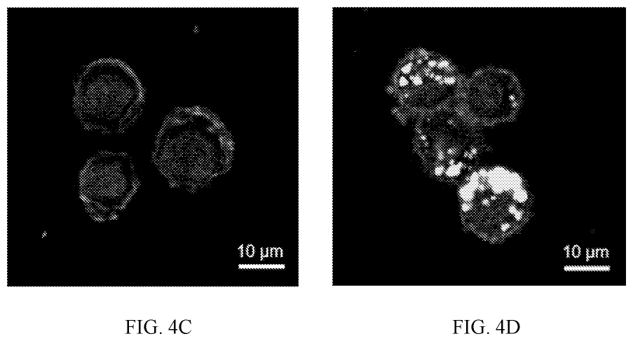

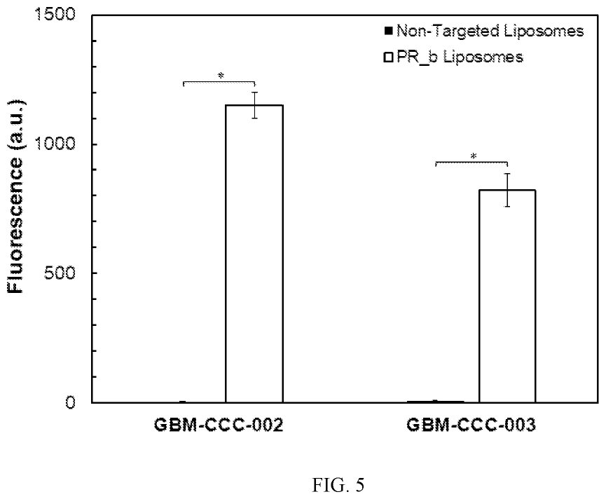

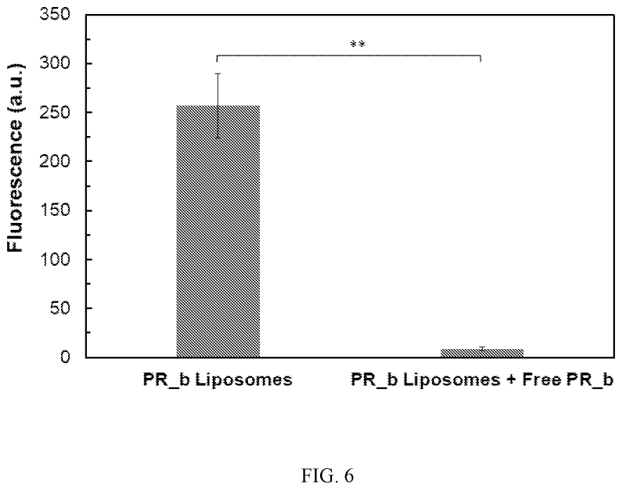

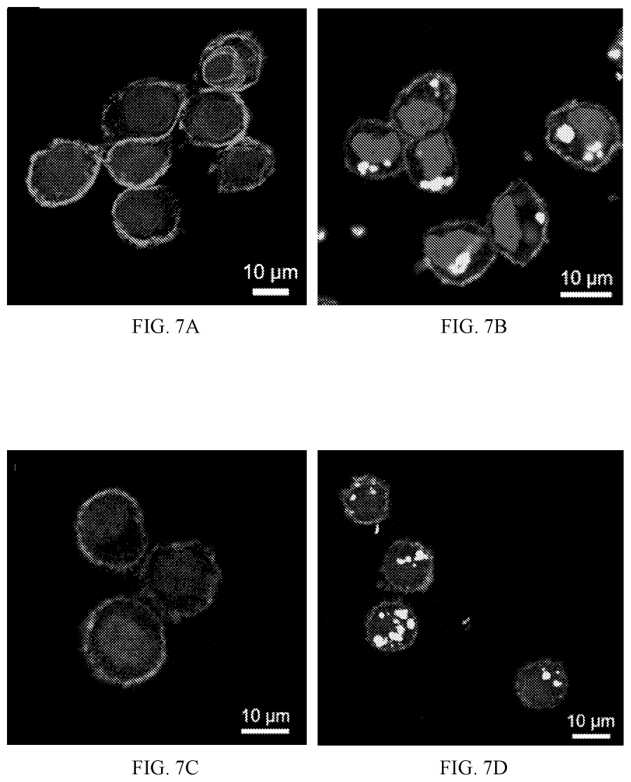

In order to enhance the intracellular accumulation of the nanoparticles with the patient-derived glioblastoma lines, the liposomes were functionalized with the PR_b peptide which binds to integrin α₅β₁ with high affinity and specificity, and is overexpressed in different types of cancer including breast, colon, prostate and pancreatic cancer. The expression of α₅β₁ was verified on GBM-CCC-001, GBM-CCC-002 and GBM-CCC-003 GSCs (FIG. 3), and FIG. 4A shows a representative flow cytometry histogram for GBM-CCC-001 cells. PR_b peptide-amphiphiles were synthesized by conjugating the PR_b peptide to a C₁₆ dialkyl tail, and were used in the preparation of the PR_b-functionalized liposomes. The association of PR_b liposomes and non-targeted liposomes with the GBM stem-like cells was investigated by flow cytometry (FIGS. 4B and 5). The fluorescence signal from the non-targeted liposomes was negligible for all glioblastoma lines tested, while the fluorescence signal from the PR_b liposomes was significantly higher than that of the non-targeted liposomes, suggesting that the PR-b targeting strongly enhances cell association. A blocking experiment with free PR_b peptide (FIG. 6) showed significant reduction in the association of the targeted liposomes with glioblastoma cells, thus demontrating specificity of the PR_b peptide for the α₅β₁ integrin. Cell internalization was further investigated with confocal microscopy. FIGS. 4C and 7 show that non-targeted liposomes did not internalize into the glioblastoma stem-like cells. In contrast, as seen in FIGS. 4D and 7, the PR_b liposomes demonstrated significant cell internalization. The confocal results are in agreement with the flow cytometry experiments and verify the role of the PR_b peptide in facilitating both the binding and internalization of PEGylated liposomes by patient-derived glioblastoma stem-like cells, thus enabling the intracellular delivery of cancer therapeutics in a selective manner.

### PR_b Liposomes Encapsulating MiR-603 PEI Complexes Suppress the Expression of IGF1 and IGF1R in Patient-Derived Glioblastoma Cells

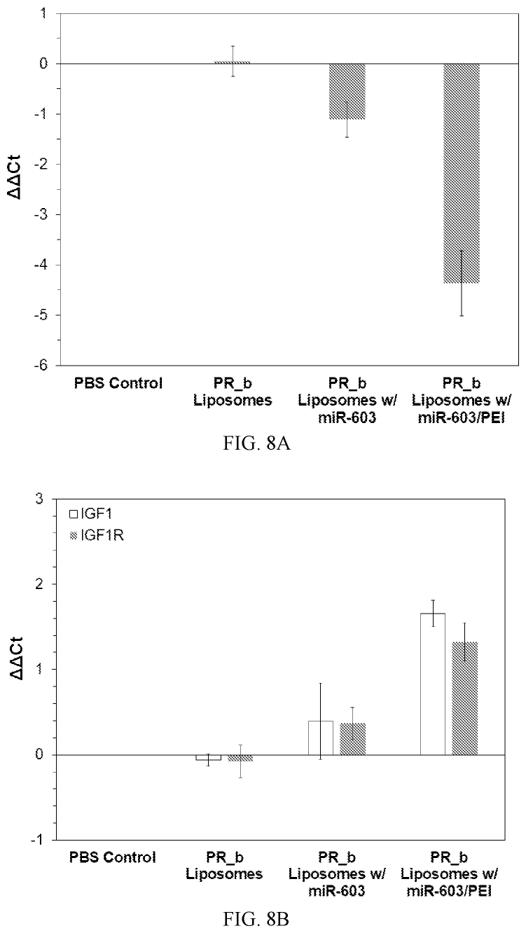

IGF1 and IGF1R are down-stream targets of miR-603. It was tested whether uptake of PR_b liposomes encapsulating miR-603/PEI complexes affected the expressions of miR-603, IGF1 and IGF1R in the GBM-CCC-001 cells. After 48 hours of treatment, PBS control, and empty PR_b liposomes had no effect on cellular levels of miR-603, IGF1 or IGF1R (FIG. 8). PR_b liposomes ecapsulating free miR-603 showed a 2.2-fold increase in cellular miR-603 level (FIG. 8A). The fold difference (1.3-fold decrease) in the IGF1 and IGF1R mRNA expression was not significant compared to the PBS control (FIG. 8B). In contrast, PR_b liposomes ecapsulating miR-603/PEI complexes induced a 22.1-fold increase in cellular miR-603 (FIG. 8A). This increase was associated with a 3.2- and 2.5-fold decrease in IGF1 and IGF1R mRNA expression, respectively (FIG. 8B). These results are generally consistent with previous reports suggesting that PEI facilitates stability and escape of cargo from liposomes and endosomes, and support our PEI-liposome platform as a therapeutic delivery strategy.

TABLE 3A
Statistical Analysis Comparing Relative miR-603 mRNA Level
in GBM-CCC-001 Cells Following Indicated Treatments^{a}

| | PR_b | PR_b liposomes | PR_b liposomes |
|---|---|---|---|
| | liposomes | w/miR-603 | w/miR-603/PEI |
| PBS control | p > 0.05 | p < 0.05 | p < 0.00001 |
| PR_b liposomes | | p < 0.05 | p < 0.00001 |
| PR_b liposomes | | | p < 0.0001 |
| w/miR-603 | | | |

^{a}P-values from one-way ANOVA with post-hoc Tukey's HSD test analysis comparing data shown in FIG. 8A (n = 3).

TABLE 3B
Statistical Analysis Comparing Relative IGF1 mRNA Level
in GBM-CCC-001 Cells Following Indicated Treatments^{a}

| | PR_b | PR_b liposomes | PR_b liposomes |
|---|---|---|---|
| | liposomes | w/miR-603 | w/miR-603/PEI |
| PBS control | p > 0.05 | p > 0.05 | p < 0.0005 |
| PR_b liposomes | | p > 0.05 | p < 0.0005 |
| PR_b liposomes | | | p < 0.001 |
| w/miR-603 | | | |

^{a}P-values from one-way ANOVA with post-hoc Tukey's HSD test analysis comparing data shown in FIG. 8B (n = 3).

TABLE 4
Statistical Analysis Comparing Relative IGF1R mRNA Level
in GBM-CCC-001 Cells Following Indicated Treatments

| | PR_b | PR_b liposomes | PR_b liposomes |
|---|---|---|---|
| | liposomes | w/miR-603 | w/miR-603/PEI |
| PBS control | p > 0.05 | p > 0.05 | p < 0.0001 |
| PR_b liposomes | | p > 0.05 | p < 0.00005 |
| PR_b liposomes | | | p < 0.005 |
| w/miR-603 | | | |

^{a} P-values from one-way ANOVA with post-hoc Tukey's HSD test analysis comparing data shown in FIG. 8B (n = 3).

### PR_b Liposomes Encapsulating MiR-603/PEI Complexes Enhance Radiation Sensitivity of Patient-Derived Glioblastoma Cells

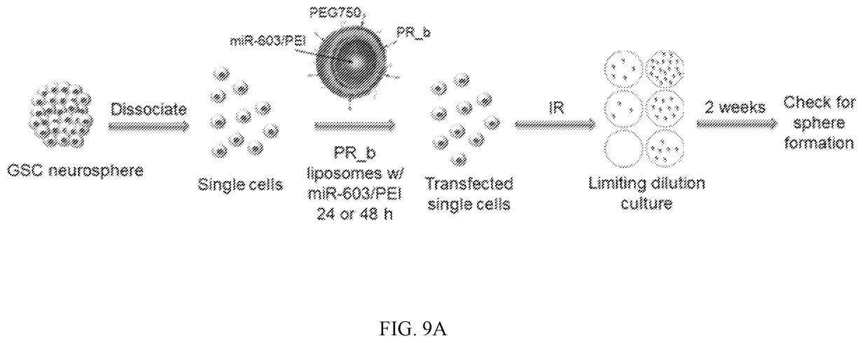

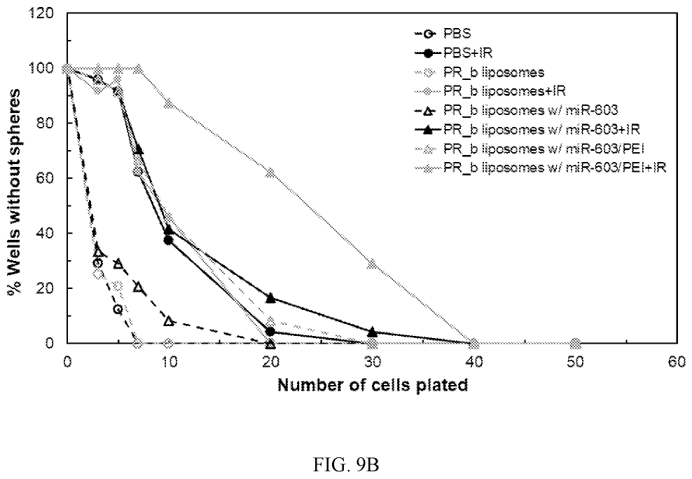

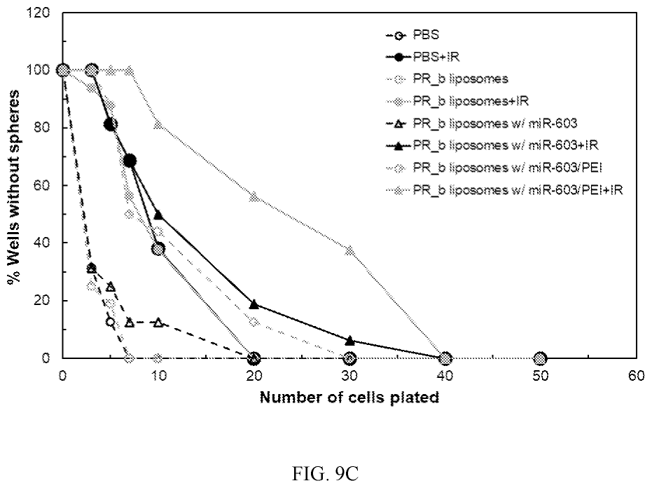

Given that PR_b liposomes encapsulating miR-603/PEI complexes suppressed the expression of IGF1 and IGF1R, it was next tested whether such treatment enhanced the IR sensitivity of patient-derived glioblastoma cells. GBM-CCC-001 were treated with PR_b liposomes encapsulating free miR-603 or miR-603/PEI complexes for 24 and 48 hours. The viabilities of these cells were assessed using a standard limiting dilution assay. In this assay, single cells were plated into 96-well plates after mock or IR treatment at various seeding densities and allowed to grow for two weeks. Higher seeding density required for sphere formation, or a rightward shift in the curve, implies decreased proliferative potential. Treatment scheme of the radiation sensitivity experiment is shown in FIG. 9A. Treatment with 6 Gy of IR resulted in an increase in the seeding density required for sphere formation, associated with a rightward shift in the proliferative potential curve. No change in seeding density requirement was observed if the radiation was carried out 24 or 48 hours after treatment with PBS, empty liposomes, or PR_b liposomes encapsulating free miR-603 (FIGS. 9B and 9C). Results suggest these treatments did not influence glioblastoma growth potential or radiation sensitivity. In contrast, treatment with PR_b liposomes encapsulating miR-603/PEI complexes for 24 or 48 hours significantly increased the seeding density required for sphere formation (shifted the sphere formation curve to the right) suggesting that the treatment enhanced radiation sensitivity (FIGS. 9B and 9C).

Quantitative analysis of the limiting dilution assay using the ELDA software is shown in Table 7. This analysis indicates that treatment with PR_b liposomes encapsulating miR-603/PEI induced a 3-fold increase in radiation sensitivity relative to treatment with PBS, empty PR_b liposomes, or PR_b liposomes encapsulating free miR-603. Similar results were observed after 24 or 48 hour incubation. Without radiation, treatment with PR_b liposomes encapsulating miR-603/PEI increased the seeding density required for sphere formation 3 to 5-fold relative to PBS, empty PR_b liposomes, or PR_b liposomes with free miR-603 for both time points. This finding is consistent with our previous results that miR-603 suppressed the glioblastoma stem cell state and the associated proliferative potential in the absence of radiation.

TABLE 5
Statistical Analysis Comparing Differences in Stem Cell Frequencies
After 24 hours Incubation with Liposomal Formulations^{a}

| | | | | | PR_b | PR_b | PR_b |
|---|---|---|---|---|---|---|---|
| | PBS | | PR_b | PR_b | liposomes | liposomes | liposomes |
| | control + | PR_b | liposomes + | liposomes | w/miR-603 + | w/miR-603/ | w/miR-603/ |
| | IR | liposomes | IR | w/miR-603 | IR | PEI | PEI + IR |
| PBS control | p < 0.00001 | p > 0.05 | p < 0.00001 | p < 0.005 | p < 0.00001 | p < 0.00001 | p < 0.00001 |
| PBS control + | | p < 0.00001 | p > 0.05 | p < 0.00001 | p > 0.05 | p > 0.05 | p < 0.00001 |
| IR | | | | | | | |
| PR_b | | | p < 0.00001 | p < 0.01 | p < 0.00001 | p < 0.00001 | p < 0.00001 |
| liposomes | | | | | | | |
| PR_b | | | | p < 0.00001 | p > 0.05 | p > 0.05 | p < 0.00001 |
| liposomes + IR | | | | | | | |
| PR_b | | | | | p < 0.00001 | p < 0.00001 | p < 0.00001 |
| liposomes w/ | | | | | | | |
| miR-603 | | | | | | | |
| PR_b | | | | | | p > 0.05 | p < 0.00001 |
| liposomes w/ | | | | | | | |
| miR-603 + IR | | | | | | | |
| PR_b | | | | | | | p < 0.00001 |
| liposomes w/ | | | | | | | |
| miR-603/PEI | | | | | | | |

^{a}P-values as calculated using the ELDA software comparing data shown in FIG. 9B (for every group, 24 wells per cell dose were used).

TABLE 6
Statistical Analysis Comparing Differences in Stem Cell Frequencies
After 48 hours Incubation with Liposomal Formulations^{a}

| | | | | | PR_b | PR_b | PR_b |
|---|---|---|---|---|---|---|---|
| | PBS | | PR_b | PR_b | liposomes | liposomes | liposomes |
| | control + | PR_b | liposomes + | liposomes | w/miR-603 + | w/miR-603/ | w/miR-603/ |
| | IR | liposomes | IR | w/miR-603 | IR | PEI | PEI + IR |
| PBS control | p < 0.00001 | p > 0.05 | p < 0.00001 | p < 0.05 | p < 0.00001 | p < 0.00001 | p < 0.00001 |
| PBS control + | | p < 0.00001 | p > 0.05 | p < 0.00001 | p > 0.05 | p > 0.05 | p < 0.00001 |
| IR | | | | | | | |
| PR_b | | | p < 0.00001 | p > 0.05 | p < 0.00001 | p < 0.00001 | p < 0.00001 |
| liposomes | | | | | | | |
| PR_b | | | | p < 0.00001 | p > 0.05 | p > 0.05 | p < 0.00001 |
| liposomes + IR | | | | | | | |
| PR_b | | | | | p < 0.00001 | p < 0.00001 | p < 0.00001 |
| liposomes w/ | | | | | | | |
| miR-603 | | | | | | | |
| PR_b | | | | | | p > 0.05 | p < 0.0005 |
| liposomes w/ | | | | | | | |
| miR-603 + IR | | | | | | | |
| PR_b | | | | | | | p < 0.00001 |
| liposomes w/ | | | | | | | |
| miR-603/PEI | | | | | | | |

^{a}P-values as calculated using the ELDA software comparing data shown in FIG. 9C (for every group, 16 wells per cell dose were used).

TABLE 7
Stem Cell Frequencies for Each Treatment.

| | 24 h | 95% CI^{b} for | 48 h | 95% CI^{b} for |
|---|---|---|---|---|
| | liposomal | 24 h | liposomal | 48 h |
| treatment^{a} | incubation | incubation | incubation | incubation |
| PBS control | 0.38 | (0.30, 0.47) | 0.37 | (0.28, 0.48) |
| PBS control + IR | 0.09 | (0.07, 0.10) | 0.09 | (0.07, 0.11) |
| PR_b liposomes | 0.35 | (0.28, 0.44) | 0.36 | (0.27, 0.47) |
| PR_b liposomes + | 0.08 | (0.07, 0.10) | 0.09 | (0.07, 0.12) |
| IR | | | | |
| PR_b liposomes | 0.23 | (0.19, 0.29) | 0.25 | (0.19, 0.32) |
| w/miR-603 | | | | |
| PR_b liposomes | 0.07 | (0.06, 0.09) | 0.07 | (0.05, 0.09) |
| w/miR-603 + IR | | | | |
| PR_b liposomes | 0.08 | (0.06, 0.10) | 0.08 | (0.06, 0.11) |
| w/miR-603/PEI | | | | |
| PR_b liposomes | 0.03 | (0.03, 0.04) | 0.03 | (0.03, 0.05) |
| w/miR-603/PEI + | | | | |
| IR | | | | |

^{a}Data are calculated with the ELDA software from measurements shown in FIGS. 9B and 9C.
^{b}Confidence intervals are reported as (lower, upper) value

These results demonstrate that targeted liposomes encapsulating miR-603 and containing a targeting moiety can be used to deliver miR-603 to GBM cells to treat the cancer.

### Example 2: Targeted Liposomes Encapsulating Anti-miR-21 Complexes Downregulate MiR-21 in Glioblastoma Cells

This Example demonstrates that targeted liposomes encapsulating anti-miR-21/PEI complexes can be used to downregulate miR-21 in glioblastoma (GBM) cells.

### Materials and Methods

### Preparation and Characterization of Liposomes

Liposomes functionalized with PR_b, encapsulating anti-miR-21/PEI complexes were prepared and characterized as discussed in Example 1. The anti-miR-21 sequence was a mixture of single-stranded DNA (ssDNA) and locked nucleic acids (LNA) that greatly enhance the stability of the anti-miR-21 and binding to the targeted miR-21:

(SEQ ID NO: 22)
5′-+T+C+AACATCAGTCTGATAA+G+C+TA-3′,

seed region underlined, LNA shown with +.

### Reverse Transcription Quantitative Real-Time Polymerase Chain Reaction (RT-qPCR)

Cells were plated at 200,000 cells per well in a 6 well plate, incubated overnight, then treated with the nanoparticles for 48 hours. Cells were trypsinized and washed twice with PBS (Phosphate Buffered Saline). TRIzol (Thermo Fisher Scientific) was added to the cells followed by absolute ethanol. mRNA was collected using a Direct-zol RNA MicroPrep mrNA isolation kit (Zymo Research). The final isolated mRNA in RNAse/DNAse free water was analyzed by UV-VIS spectrometry using a Synergy H1 plate reader (BioTek). The mRNA was diluted using MilliQ water to 5 ng/μL, and cDNA synthesis was completed using a miRCURY LNA RT kit (Qiagen). For this cDNA synthesis the mRNA was combined with 5× miRCURY RT SYBR green reaction buffer, 10× miRCURY RT Enzyme mix, UniSP6 RNA Spike-in Template, and MilliQ and thermo-cycled according to manufacturer instructions. This cDNA was combined with miRCURY LNA miRNA PCR assays, 2× miRCURY SYBR green master mix, and MilliQ water according to manufacturer instructions and the polymerase chain reaction (PCR) was performed with CFX384 Touch Real-Time PCR Detection System (Bio-Rad). miR-21 expression was normalized to the UniSP6 spike in expression and compared to the untreated control.

### Results and Discussion

Liposomes functionalized with the targeting PR_b peptide and encapsulating anti-miR-21/PEI complexes were prepared and characterized as described in Example 1. The nanoparticles had an average diameter of 123 nm with a PDI of 0.078 and a zeta potential of 21.1 mV. The final concentration of PR_b on the liposome surface was 2.71 mol %. The encapsulation efficiency of the anti-miR-21/PEI complexes was around 78%.

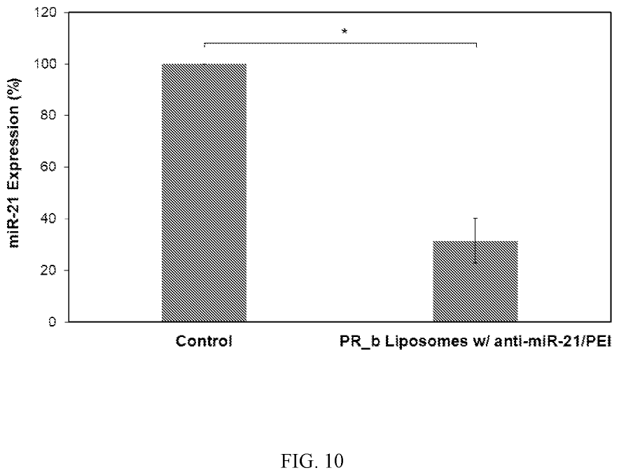

The targeted liposomes were incubated with U87 human GBM cells for 48 hours. At the end of the experiment, the expression of miR-21 at the mRNA level relative to the control (untreated cells) was evaluated with RT-qPCR. FIG. 10 shows that the targeted liposomes could effectively down-regulate the expression of the oncogenic miR-21 in U87 cells.

### Example 3: Targeted Lipid Nanoparticles (LNPs) Encapsulating Anti-miR-21 or MiR-603 Decrease Cancer Cell Proliferation

This Example demonstrates that targeted LNPs encapsulating anti-miR-21 or miR-603 can significantly decrease the proliferation of different cancer cells.

### Materials and Methods

### Cell Culture

Human GBM cells (U87, A172), and human triple negative breast cancer (TNBC) cells (MDA-MB-231) were cultured at 37° C. and 5% CO₂ in T75 flasks in Dulbecco's modified Eagle medium (DMEM) (Thermo Fisher Scientific) supplemented with 10% (v/v) heat-inactivated fetal bovine serum (FBS) (Thermo Fisher Scientific) and 1% (v/v) penicillin/streptomycin (penicillin 100 U/mL and streptomycin 0.1 mg/mL final concentrations) (MilliporeSigma). Human pancreatic cancer cells (PANC 10.05) were cultured at 37° C. and 5% CO₂ in T75 flasks in RPMI 1640 medium (Thermo Fisher Scientific) supplemented with 10% (v/v) heat-inactivated FBS, 0.2 U/mL recombinant human insulin (MP Biomedical), and 1% (v/v) penicillin/streptomycin (penicillin 100 U/mL and streptomycin 0.1 mg/mL final concentrations) (MilliporeSigma). Upon reaching greater than 80% confluence, cells were washed twice with 1×PBS and detached using 0.05% trypsin-EDTA (Corning). Cells were then pelleted via centrifugation, resuspended in fresh media, and plated as needed.

### Preparation and Characterization of PR_b LNPs

For the preparation of the PR_b functionalized LNPs, SM-102 (Echelon Biosciences), PEG2000-DMG (Avanti Polar Lipids), DSPC (Avanti Polar Lipids), β-sitosterol (MedChemExpress) and PR_b-amphiphile (synthesized as in Example 1) were combined at SM-102:PEG2000-DMG:DSPC:β-sitosterol:PR_b-amphiphile 50:1.5:10:33.5:5 mol %. The targeted LNPs were prepared via two different methods: 1) by mixing the PR_b peptide-amphiphile in the buffer (referred as PR_b (mix) LNP), or by post-inserting the peptide-amphiphile in preformed LNPs (referred as PR_b (pi) LNP). For the PR_b (mix) LNP, the PR_b-amphiphile was combined with the anti-miR-21 (same sequence as in Example 2) or miR-603 in 50 mM sodium acetate buffer pH=4.0. All other components of the LNP were added in pure ethanol, with the SM-102 at a 7:1 nitrogen to phosphate (N:P) ratio, where the phosphate (P) is evaluated from the anti-miR or miR sequence. The total volume of the aqueous buffer was three times that of the ethanol solution. For the PR_b (pi) LNP, the buffer (50 mM sodium acetate buffer pH=4.0) only contained the miR-603. The ethanol solution contained the SM-102, PEG2000-DMG, DSPC and B-sitosterol, with the SM-102 at a 7:1 N:P ratio (where the P is evaluated from the miR). The total volume of the aqueous buffer was three times that of the ethanol solution.

Syringe pumps were used to combine the two solutions in a Fluidic 187 Herringbone Mixer chip (Microfluidic ChipShop) with the buffer pumped at 2.25 mL/minute and the ethanol solution pumped at 0.75 mL/minute. The collected LNPs were then immediately dialyzed overnight against 1 L 1×PBS using a 3.5 kDa MWCO Pur-A-Lyzer dialysis tube. Following dialysis, the LNPs were sterile filtered using a 0.2 μm syringe filter. For the PR_b (pi) LNPs the sterile filtered LNPs were combined with the PR_b-amphiphile and shaken at 400 rpm at 37° C. overnight. These PR_b (pi) LNPs were then dialyzed overnight against 1 L 1×PBS using a 3.5 kDa MWCO Pur-A-Lyzer dialysis tube.

The final concentration of PR_b on the surface of the LNPs was evaluated using a BCA protein assay (Thermo Fisher Scientific) following the manufacturer's protocol with a standard curve based on the PR_b peptide-amphiphile. The size and zeta potential of the LNPs were measured using a Zetasizer (Malvern Panalytical). The encapsulation efficiency for the anti-miR-21 or miR-603 in the LNPs was determined by diluting to 5 ng/μL in either 1×TE buffer or 1×TE buffer with 1% triton X-100. 10 μL of this solution was then combined with 190 μL 1×TE buffer and 10 μL of 1 μM DiTO dye in 1×TE in a black 96-well flat bottom plate. The plate was shaken for 30 seconds, incubated in the dark for 5 minutes, and the fluorescence was read. The measured fluorescence was used to determine the total amount (with triton) of anti-miR or miR as well the unencapsulated amount (no triton). These values were then used to determine the encapsulation efficiency.

### Cryogenic Transmission Electron Microscopy (Cryo-TEM)

The morphology of the PR_b LNPs encapsulating anti-miR-21 or miR-603 was examined via cryo-TEM. 5 μL of the LNP solution were deposited onto lacey formvar/carbon copper grids (Ted Pella) that had been treated with glow discharge for 30 seconds and vitrified in liquid ethane by Vitrobot (Vitrobot parameters: 4 second blot time, 0 offset, 3 second wait time, 100% humidity). After vitrification, the grids were kept under liquid nitrogen and were transferred to a F200C Talos TEM operated at an acceleration voltage of 200 kV (Integrated Imaging Center at the Johns Hopkins University, Institute for NanoBioTechnology). Images were captured using the Ceta camera of the Talos.

### Reverse Transcription Quantitative Real-Time Polymerase Chain Reaction (RT-qPCR)

RT-qPCR was done as described in Example 2.

### Cell Proliferation Study

Cells were plated at 5,000 cells per well in white 96-well plates and incubated for about 24 hours. The next day the media was replaced and the PR_b LNPs were spiked in anti-miR-21 or miR-603 concentration of 90 nM and 180 nM. The cells were then incubated for 48 hours before analyzing by CellTiter Glo 2.0 according to manufacturer's instructions.

### Results and Discussion

The PR_b (mix) LNP encapsulating anti-miR-21 had an average diameter of 76.59±2.44 nm with a PDI of 0.183 and a zeta potential of 10.89±1.28 mV. The final concentration of PR_b on the liposome surface was 4.57 mol %. The encapsulation efficiency of anti-miR-21 was around 86%.

The PR_b (mix) LNP encapsulating miR-603 had an average diameter of 115 nm with a PDI of 0.22 and a zeta potential of 7.85±5.05 mV. The final concentration of PR_b on the liposome surface was around 5 mol %. The encapsulation efficiency of miR-603 was >65%.

The PR_b (pi) LNP encapsulating miR-603 had an average diameter of 92.65 nm with a PDI of 0.17 and a zeta potential of 14.17±6.72 mV. The final concentration of PR_b on the liposome surface was around 4.94 mol %. The encapsulation efficiency of miR-603 was 86%.

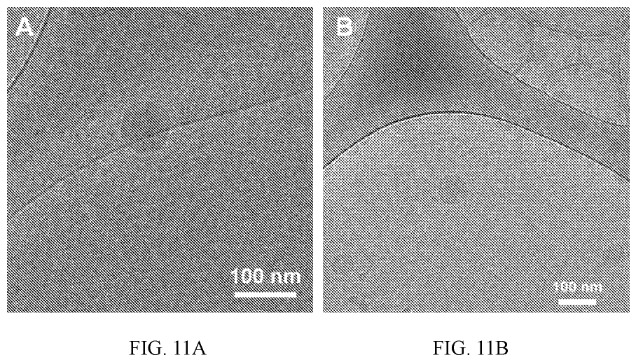

Cryo-TEM images of PR_b (mix) LNPs encapsulating anti-miR-21 (FIG. 11A) and miR-603 (FIG. 11B) verified the round morphology of the LNPs.

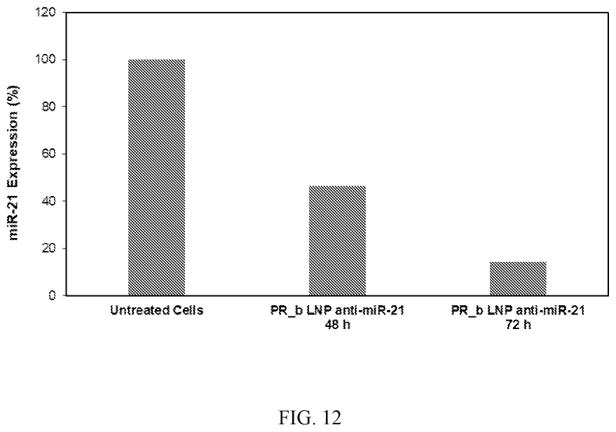

The ability of the PR_b (mix) LNPs encapsulating anti-miR-21 to downregulate miR-21 expression at the mRNA level was evaluated with PANC 10.05 human pancreatic cancer cells. FIG. 12 shows that the LNPs successfully downregulated the oncogenic miR-21. The level of downregulation increased with increased incubation of the LNPs with the cancer cells, with only 14.34% of miR-21 expressed after 72 h compared to the untreated (control) cells.

In addition, PR_b (mix) LNPs encapsulating 40 nM miR-603 were delivered to U87 GBM cells, and after 48 hours of incubation the expression of miR-603 at the mRNA level was increased by 22,851-fold (ΔΔCt=−14.5).

The ability of the PR_b LNPs to decrease the proliferation of different cancer cells was evaluated next. Table 8 shows the effect of PR_b (mix) LNP encapsulating anti-miR-21 at 90 nM at 180 nM after 48 hour incubation with GBM and TNBC cells. Table 9 shows the effect of PR_b (mix) LNP and PR_b (pi) LNP encapsulating miR-603 at 90 nM at 180 nM after 48 hour incubation with GBM and TNBC cells. Tables 8 and 9 show that the PR_b LNPs encapsulating anti-miR-21 or miR-603 significantly decrease the viability of different cancer cells and the cytotoxicity of these nanoparticles increases with increased miR or anti-miR concentration.

TABLE 8
Cell viability of different cancer cells after 48 hour incubation with
PR_b LNPs encapsulating different concentrations of anti-miR-21.

| | | Cell |
|---|---|---|
| Cell Type | Treatment | Viability (%) |
| All cells | No Treatment (Control) | 100 |
| U87 | PR_b (mix) LNP anti-miR-21 (90 nM) | 80.6 |
| | PR_b (mix) LNP anti-miR-21 (180 nM) | 27.2 |
| MDA-MB-231 | PR_b (mix) LNP anti-miR-21 (90 nM) | 83.9 |
| | PR_b (mix) LNP anti-miR-21 (180 nM) | 67.3 |

TABLE 9
Cell viability of different cancer cells after 48 hour incubation
with PR_b LNPs encapsulating different concentrations of miR-603.

| | | Cell |
|---|---|---|
| Cell Type | Treatment | Viability (%) |
| All cells | No Treatment (Control) | 100 |
| U87 | PR_b (mix) LNP miR-603 (90 nM) | 13.9 |
| | PR_b (mix) LNP miR-603 (180 nM) | 0.6 |
| | PR_b (pi) LNP miR-603 (90 nM) | 28.7 |
| | PR_b (pi) LNP miR-603 (180 nM) | 4.0 |
| A172 | PR_b (mix) LNP miR-603 (90 nM) | 62.3 |
| | PR_b (mix) LNP miR-603 (180 nM) | 0.2 |
| | PR_b (pi) LNP miR-603 (90 nM) | 60.2 |
| | PR_b (pi) LNP miR-603 (180 nM) | 2.8 |
| MDA-MB-231 | PR_b (mix) LNP miR-603 (90 nM) | 68.5 |
| | PR_b (mix) LNP miR-603 (180 nM) | 52.3 |
| | PR_b (pi) LNP miR-603 (90 nM) | 68.4 |
| | PR_b (pi) LNP miR-603 (180 nM) | 25.9 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nanoparticle encapsulating a microRNA, wherein said nanoparticle comprises a targeting moiety comprising a polypeptide comprising the amino acid sequence set forth in SEQ ID NO:5.

2. The nanoparticle of claim 1, wherein said microRNA is selected from the group consisting of miR-603, miR-34a, miR-218, miR-219, miR-183m, miR-451, miR-133, miR-134, miR-302c, miR-324, miR-379, miR-491, miR-340, miR-7, miR-128, miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, a microRNA in the miR-200 family, miR-203, miR-203b-3p, miR-205, miR-206, miR-217, miR-298, miR-375, miR-490-3p, miR-539, miR-542-3p, miR-613, miR-638, miR-940, and a microRNA in the let-7 family.

3. The nanoparticle of claim 2, wherein said microRNA is miR-603.

4. The nanoparticle of claim 1, wherein said nanoparticle is selected from the group consisting of a liposome, a lipid nanoparticle, an extracellular vesicle, a polymersome, a polymeric nanoparticle, and a micelle.

5. (canceled)

6. (canceled)

7. The nanoparticle of claim 1, wherein said targeting moiety is conjugated to a hydrophobic tail having the structure (C₁₆)₂-Glu-C₂.

8. A nanoparticle encapsulating an anti-microRNA, wherein said nanoparticle comprises a targeting moiety comprising a polypeptide comprising the amino acid sequence set forth in SEQ ID NO:5.

9. The nanoparticle of claim 8, wherein said anti-microRNA is selected from the group consisting of anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b-5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, and anti-miR-1908.

10. The nanoparticle of claim 9, wherein the anti-microRNA is anti-miR-21.

11. The nanoparticle of claim 8, wherein said nanoparticle is selected from the group consisting of a liposome, a lipid nanoparticle, an extracellular vesicle, a polymersome, a polymeric nanoparticle, and a micelle.

12. (canceled)

13. (canceled)

14. The nanoparticle of claim 8, wherein said targeting moiety is conjugated to a hydrophobic tail having the structure (C₁₆)₂-Glu-C₂.

15. A method for treating a mammal having cancer, wherein said method comprises:
administering a composition comprising the nanoparticle of claim 1 to said mammal.

16. The method of claim 15, wherein said mammal is a human.

17. The method of claim 15, wherein said cancer is selected from the group consisting of a glioblastoma, a diffuse intrinsic pontine glioma, an astrocytoma, an oligodendroglioma, an oligoastrocytoma, an ependymoma, a medulloblastoma, a meningioma, a breast cancer, a colon cancer, a liver cancer, a pancreatic cancer, and a prostate cancer.

18. (canceled)

19. (canceled)

20. A method for sensitizing cancer cells to radiation therapy, wherein said method comprises:
administering a composition comprising the nanoparticle of claim 1 to a mammal having cancer.

21. The method of claim 20, wherein said mammal is a human.

22. The method of claim 20, wherein said cancer is selected from the group consisting of a glioblastoma, a diffuse intrinsic pontine glioma, an astrocytoma, an oligodendroglioma, an oligoastrocytoma, an ependymoma, a medulloblastoma, a meningioma, a breast cancer, a colon cancer, a liver cancer, a pancreatic cancer, and a prostate cancer.

23. (canceled)

24. (canceled)

25. A method for treating a mammal having cancer, wherein said method comprises:
administering a composition comprising the nanoparticle of claim 1 to said mammal; and
subjecting said mammal to radiation therapy.

26. The method of claim 25, wherein said mammal is a human.

27. The method of claim 25, wherein said cancer is selected from the group consisting of a glioblastoma, a diffuse intrinsic pontine glioma, an astrocytoma, an oligodendroglioma, an oligoastrocytoma, an ependymoma, a medulloblastoma, a meningioma, a breast cancer, a colon cancer, a liver cancer, a pancreatic cancer, and a prostate cancer.

28. (canceled)


SEQUENCE LISTING (22 sequences)

SEQ ID NO: 1 - RNA, 22 nt, Artificial
  miR-603 microRNA sequence
cacacacugcaauuacuuuugc

SEQ ID NO: 2 - RNA, 22 nt, Artificial
  miR-603 microRNA sequence
aaaaguaauugcaguguguguu

SEQ ID NO: 3 - protein, 5 aa, Artificial
  polypeptide that can be included in a targeting moiety that can bind integrin alpha 5 beta 1 polypeptides
RGDSP

SEQ ID NO: 4 - protein, 5 aa, Artificial
  polypeptide that can be included in a targeting moiety that can bind integrin alpha5beta1 polypeptides
PHSRN

SEQ ID NO: 5 - protein, 23 aa, Artificial
  polypeptide that can be included in a targeting moiety that can bind integrin alpha5beta1 polypeptides
KSSPHSRNSGSGSGSGSGRGDSP

SEQ ID NO: 6 - protein, 18 aa, Artificial
  polypeptide that can be included in a targeting moiety that can bind integrin alpha5beta1 polypeptides
PHSRNSGSGSGSGRGDSP

SEQ ID NO: 7 - protein, 23 aa, Artificial
  polypeptide that can be included in a targeting moiety that can bind integrin alpha5beta1 polypeptides
KSSPHSRNGGGGGGGGGGRGDSP

SEQ ID NO: 8 - protein, 24 aa, Artificial
  polypeptide that can be included in a targeting moiety that can bind integrin alpha5beta1 polypeptides
KSSPHSRNSSSSSSSSSSGRGDSP

SEQ ID NO: 9 - protein, 20 aa, Artificial
  polypeptide that can be included in a targeting moiety that can bind integrin alpha5beta1 polypeptides
PHSRNSGSGSGSGSGRGDSP

SEQ ID NO: 10 - protein, 26 aa, Artificial
  polypeptide that can be included in a targeting moiety that can bind integrin alpha5beta1 polypeptides
KSSSSSPHSRNSGSGSGSGSGRGDSP

SEQ ID NO: 11 - DNA, 20 bp, Artificial
  synthetic oligonucleotide primer
gcagcactcatccacgatgc

SEQ ID NO: 12 - DNA, 24 bp, Artificial
  synthetic oligonucleotide primer
tgtggagacaggggcttttatttc

SEQ ID NO: 13 - DNA, 19 bp, Artificial
  synthetic oligonucleotide primer
aagttctggttgtcgagga

SEQ ID NO: 14 - DNA, 21 bp, Artificial
  synthetic oligonucleotide primer
gagcagctagaagggaattac

SEQ ID NO: 15 - DNA, 19 bp, Artificial
  synthetic oligonucleotide primer
ttgccctccaatggatcct

SEQ ID NO: 16 - DNA, 26 bp, Artificial
  synthetic oligonucleotide primer
gggaggtagtgacgaaaaataacaat

SEQ ID NO: 17 - protein, 10 aa, Artificial
  linker that can be included in a targeting moiety that can bind integrin alpha5beta1 polypeptides
SGSGSGSGSG

SEQ ID NO: 18 - protein, 8 aa, Artificial
  linker that can be included in a targeting moiety that can bind integrin alpha5beta1 polypeptides
SGSGSGSG

SEQ ID NO: 19 - protein, 10 aa, Artificial
  linker that can included in a targeting moiety that can bind integrin alpha5beta1 polypeptides
GGGGGGGGGG

SEQ ID NO: 20 - protein, 10 aa, Artificial
  linker that can be included in a targeting moiety that can bind integrin alpha5beta1 polypeptides
SSSSSSSSSS

SEQ ID NO: 21 - protein, 6 aa, Artificial
  polypeptide that can be included in a targeting moiety that can bind integrin alpha5beta1 polypeptides
GRGDSP

SEQ ID NO: 22 - DNA, 22 bp, Artificial
  anti-miR-21 anti-microRNA sequence
tcaacatcagtctgataagcta